SEQUENCE LISTING (17 sequences)

SEQ ID NO: 1 - protein, 135 aa, Mus musculus
MEWSRVFIFLLSVTAGVHSQVQLQQSGAELVRPGTSVKVSCKASGYAFTYYLIEWVKQRPGQGLEWIGVINPGSGGTNYNEKFKGKATLTADKSSSTAYMQLSSLTSDDSAVYFCARNWMNFDYWGQGTTLTVSS

SEQ ID NO: 2 - protein, 132 aa, Mus musculus
MRCLAEFLGLLVLWIPGAIGDIVMTQAAPSVSVTPGESVSISCRSSKSLLHSNGNTYLYWFLQRPGQSPQFLIYRMSNLASGVPDRFSGSGSGTAFTLRISRVEAEDVGVYYCMQHLEYPYTFGGGTKLEIK

SEQ ID NO: 3 - protein, 116 aa, Artificial Sequence
  synthetic construct
QVQLVQSGAEVKKPGASVKVSCKASGYAFTYYLIEWVRQAPGQGLEWIGVINPGSGGTNYNEKFKGRATITADKSTSTAYMELSSLRSEDTAVYFCARNWMNFDYWGQGTTVTVSS

SEQ ID NO: 4 - protein, 112 aa, Artificial Sequence
  synthetic construct
DIVMTQTPLSLSVTPGQPASISCRSSKSLLHSNGNTYLYWFLQKPGQSPQFLIYRMSNLASGVPDRFSGSGSGTDFTLKISRVEAEDVGVYYCMQHLEYPYTFGGGTKVEIK

SEQ ID NO: 5 - DNA, 405 bp, Mus musculus
atggaatggagcagagtctttatctttctcctatcagtaactgcaggtgttcactcccaggtccagttgcagcagtctggagctgagctggtaaggcctgggacttcagtgaaggtgtcctgcaaggcttctggatacgccttcacttattacttgatagagtgggtaaagcagaggcctggacagggccttgagtggattggggtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaaggaactggatgaactttgactactggggccaaggcaccactctcacagtctcctca

SEQ ID NO: 6 - DNA, 396 bp, Mus musculus
atgaggtgcctagctgagttcctggggctgcttgtgctctggatccctggagccattggggatattgtgatgactcaggctgcaccctctgtatctgtcactcctggagagtcagtatccatctcctgcaggtctagtaagagtctcctgcatagtaatggcaacacttacttgtattggttcctgcagaggccaggccagtctcctcagttcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccttacacgttcggaggggggaccaagctggaaataaaa

SEQ ID NO: 7 - DNA, 348 bp, Artificial Sequence
  synthetic construct
caggtgcagctggtgcagtctggggctgagctgaagaagcctggggcctcagtgaaggtctcctgcaaggcttctggatacgccttcacttattacttgatagagtgggtgaaacaggcccctggacaagggcttgagtggatcggagtgattaatcctggaagtggtggtactaactacaatgagaagttcaagggcagagccacgctcaccgcggacaaatccacgagcacagcctacatggagctgagcagcctgagatctgaggactccgccgtgtatttctgtgcgagaaactggatgaactttgactactgggggcaagggaccacggtcaccgtctcctca

SEQ ID NO: 8 - DNA, 348 bp, Artificial Sequence
  synthetic construct
caggtgcagctggtgcagtctggggctgaggtgaagaagcctggggcctcagtgaaggtctcctgcaaggcttctggatacgccttcacttattacttgatagagtgggtgagacaggcccctggacaagggcttgagtggatcggagtgattaatcctggaagtggtggtactaactacaatgagaagttcaagggcagagccacgctcaccgcggacaaatccacgagcacagcctacatggagctgagcagcctgagatctgaggacaccgccgtgtatttctgtgcgagaaactggatgaactttgactactgggggcaagggaccacggtcaccgtctcctca

SEQ ID NO: 9 - DNA, 348 bp, Artificial Sequence
  synthetic construct
caggtgcagctggtgcagtctggggctgaggtgaagaagcctggggcctcagtgaaggtctcctgcaaggcttctggatacgccttcacttattacttgatagagtgggtgagacaggcccctggacaagggcttgagtggatcggagtgattaatcctggaagtggtggtactaactacaatgagaagttcaagggcagagccacgatcaccgcggacaaatccacgagcacagcctacatggagctgagcagcctgagatctgaggacaccgccgtgtatttctgtgcgagaaactggatgaactttgactactgggggcaagggaccacggtcaccgtctcctca

SEQ ID NO: 10 - DNA, 348 bp, Artificial Sequence
  synthetic construct
caggtgcagctggtgcagtctggggctgaggtgaagaagcctggggcctcagtgaaggtctcctgcaaggcttctggatacgccttcacttattacttgatagagtgggtgagacaggcccctggacaagggcttgagtggatcggagtgattaatcctggaagtggtggtactaactacaatgagaagttcaagggcagagtcacgatcaccgcggacaaatccacgagcacagcctacatggagctgagcagcctgagatctgaggacaccgccgtgtattactgtgcgagaaactggatgaactttgactactgggggcaagggaccacggtcaccgtctcctca

SEQ ID NO: 11 - DNA, 336 bp, Artificial Sequence
  synthetic construct
gatattgtgatgacccagactccactctctctgtccgtcacccctggacagccggcctccatctcctgcaggtctagtaagagtctcctgcatagtaatggcaacacttacttgtattggttcctgcagaagccagggcagtctccacagttcctgatctatcggatgtccaaccttgcctcaggagtgccagataggttcagtggcagcgggtcagggacagccttcacactgaaaatcagccgggtggaggctgaggatgttggggtttattactgcatgcaacatctagaatatccttacaccttcggcggagggaccaaggtggagatcaaa

SEQ ID NO: 12 - DNA, 336 bp, Artificial Sequence
  synthetic construct
gatattgtgatgacccagactccactctctctgtccgtcacccctggacagccggcctccatctcctgcaggtctagtaagagtctcctgcatagtaatggcaacacttacttgtattggttcctgcagaagccagggcagtctccacagttcctgatctatcggatgtccaaccttgcctcaggagtgccagataggttcagtggcagcgggtcagggacagacttcacactgaaaatcagccgggtggaggctgaggatgttggggtttattactgcatgcaacatctagaatatccttacaccttcggcggagggaccaaggtggagatcaaa

SEQ ID NO: 13 - DNA, 336 bp, Artificial Sequence
  synthetic construct
gatattgtgatgacccagactccactctctctgtccgtcacccctggacagccggcctccatctcctgcaggtctagtaagagtctcctgcatagtaatggcaacacttacttgtattggtacctgcagaagccagggcagtctccacagttcctgatctatcggatgtccaaccttgcctcaggagtgccagataggttcagtggcagcgggtcagggacagacttcacactgaaaatcagccgggtggaggctgaggatgttggggtttattactgcatgcaacatctagaatatccttacaccttcggcggagggaccaaggtggagatcaaa

SEQ ID NO: 14 - RNA, 25 nt, Artificial Sequence
  synthetic construct
uggaguaaucggauucugcaaccuc

SEQ ID NO: 15 - RNA, 25 nt, Artificial Sequence
  synthetic construct
ucaacgaauugucaaauuugaaccc

SEQ ID NO: 16 - DNA, 58 bp, Artificial Sequence
  synthetic construct
ccggcgattactccaacaacatcatctcgagatgatgttgttggagtaatcgtttttg

SEQ ID NO: 17 - DNA, 58 bp, Artificial Sequence
  synthetic construct
ccgggaaggagacatccagaagaatctcgagattcttctggatgtctccttctttttg

(19) United States
(12) Patent Application Publication
(10) Pub. No.: US 2014/0186340 A1
(43) Pub. Date: Jul. 3, 2014

(54) Methods and Compositions for Normalization of Tumor Vasculature by Inhibition of LOXL2

(71) Applicants: Derek Marshall, Foster City, CA (US); Rhyannon Spangler, Foster City, CA (US); Victoria Smith, Foster City, CA (US)

(72) Inventors: Derek Marshall, Foster City, CA (US); Rhyannon Spangler, Foster City, CA (US); Victoria Smith, Foster City, CA (US)

(73) Assignee: GILEAD BIOLOGICS, INC., Foster City, CA (US)

(21) Appl. No.: 14/110,710

(22) PCT Filed: Apr. 6, 2012
(86) PCT No.: PCT/US12/32600

§ 371 Date: Jan. 21, 2014

Related U.S. Application Data
(60) Provisional application No. 61/473,628, filed on Apr. 8, 2011.

(51) Int. Cl. C07K 16/40; C12N 15/113

(52) U.S. Cl. CPC C07K 16/40; C12N 15/1137
USPC 424/133.1; 514/44 A

(57) ABSTRACT

Disclosed herein are methods and compositions for increasing perfusion, reducing hypoxia, reducing permeability and increasing the integrity of vasculature; e.g., in a tumor. The compositions include inhibitors of the LOXL2 enzyme. In certain of the methods, a LOXL2 inhibitor is used in combination with, and facilitates the therapeutic activity of, an anti-neoplastic agent.

### CROSS REFERENCE TO RELATED APPLICATIONS

This application claims benefit of U.S. provisional application No. 61/473,628, filed on Apr. 8, 2011, the disclosure of which is hereby incorporated by reference in its entirety for all purposes.

### STATEMENT REGARDING FEDERAL SUPPORT

Not applicable.

### REFERENCE TO SEQUENCE LISTING SUBMITTED VIA EFS-WEB

The entire content of the following electronic submission of the sequence listing via the USPTO EFS-WEB server, as authorized and set forth in MPEP §1730 II.B.2(a)(C), is incorporated herein by reference in its entirety for all purposes. The sequence listing is identified on the electronically filed text file as follows:

| File Name | Date of Creation | Size (bytes) |
|---|---|---|
| 246102008140Seqlist.txt | Apr. 5, 2012 | 10,383 bytes |

### FIELD

This disclosure is in the field of vascular biology and angiogenesis, particularly tumor angiogenesis.

### BACKGROUND

Solid tumors require a blood supply to grow and, eventually to metastasize. Tumor vasculature, however, is abnormal in the sense that it is dense, disorganized, saccular, dilated, haphazardly interconnected, more tortuous than normal vasculature and more permeable than normal vasculature (i.e., leaky). Tumor vessels are also characterized by morphologically abnormal endothelial cells lining the vessels, fewer pericytes (endothelial supporting cells) and an aberrant basement membrane, which can be abnormally thick or absent altogether.

Because of the properties of its vasculature, blood flow in a tumor is both spatially and temporally non-uniform. Furthermore, tumor growth exerts pressure that compresses blood and lymphatic vessels, further impairing blood flow through the tumor. As a result, the abnormal vasculature of a tumor is characterized by an inability to perfuse all regions of the tumor and by leakiness of vessels in the perfused regions. Consequently, tumor vasculature is poor at delivering chemotherapeutics and anti-neoplastic agents uniformly throughout a tumor.

Because of their poor vasculature, increased extravascular pressure and other factors, tumors contain lower-than-physiological levels of oxygen (i.e., are hypoxic). Tumor hypoxia lessens the effectiveness of many chemotherapeutics and also compromises radiation treatment, which relies, in part, on generation of reactive oxygen radicals.

Inhibition of angiogenesis and/or vasculogenesis in tumors might be expected to inhibit tumor growth; but could also be counterproductive by further reducing the delivery of anti-neoplastic agents (e.g., chemotherapeutic drugs) and oxygen to a tumor. Increased tumor hypoxia resulting from a total loss of vasculature would also be expected to further lessen the effectiveness of chemotherapeutics and radiation treatment.

To facilitate the delivery (and, in some cases, the therapeutic activity) of anti-neoplastic agents (e.g., chemotherapeutic drugs) and to enhance the efficacy of radiation treatments, it would be desirable to have methods and compositions capable of returning tumor vasculature to a more normal state, so that the tumor would be more uniformly perfused with non-leaky vessels.

Therefore, there is a need for methods and compositions that are able to alter tumor vasculature without destroying it. In particular, methods and compositions for transiently normalizing tumor vasculature, to facilitate the delivery and/or action of anti-neoplastic agents, are desirable.

### SUMMARY

In some aspects, the provided embodiments are based on the discovery that inhibition of LOXL2 activity, in a tumor, reduces the degree of vascularization of the tumor, but that the reduced vasculature is more physiologically normal and provides better perfusion of the tumor. Such inhibition is shown herein to potentiate the effects of chemotherapeutic drugs, leading to more effective inhibition of tumor growth.

Accordingly, the present disclosure provides, inter alia, the following embodiments:

Provided are methods for normalizing vasculature in a tumor, methods for reducing hypoxia in a tumor, methods for increasing perfusion in a tumor, methods for facilitating the delivery of a chemotherapeutic to a tumor, methods for increasing the efficacy of an anti-neoplastic agent, for example, in reducing tumor growth, and methods for increasing the sensitivity of a tumor to a chemotherapeutic, and combinations thereof. Such methods in certain aspects are carried out by administering to a subject with the tumor an inhibitor of LOXL2. In some examples, normalizing comprises conversion of dense, leaky vasculature to a less dense system of vessels that are less permeable. In some examples, normalizing effects an increase in the number of pericytes associated with endothelial cells. In some examples, the normalized vessels are less tortuous and/or are less dilated.

In some aspects, for example, where the method is for facilitating delivery of a chemotherapeutic to the tumor or for increasing the sensitivity of a tumor to a chemotherapeutic, the chemotherapeutic is selected from the group consisting of a chemotherapeutic drug and a therapeutic biologic.

In some aspects, for example, where the method is for increasing efficacy of an anti-neoplastic agent, e.g., in reducing tumor growth, the anti-neoplastic agent is selected from the group consisting of a chemotherapeutic drug, a therapeutic biologic and radiation.

In some embodiments, the inhibitor of LOXL2 is an anti-LOXL2 antibody or antigen-binding fragment thereof. In some examples, the anti-LOXL2 antibody or fragment has a heavy-chain amino acid sequence as set forth in SEQ ID NO:3, or and/or has a light-chain amino acid sequence as set forth in SEQ ID NO:4, or an antigen-binding portion thereof. In some examples, the anti-LOXL2 antibody or fragment has an one or more CDRs (e.g., CDR1, CDR2, and/or CDR3) of a heavy chain amino acid sequence as set forth in SEQ ID NO: 1 or 3, and/or has one or more CDRs (e.g., CDR1, CDR2, and/or CDR3) of a light chain amino acid sequence as set forth in SEQ ID NO: 2 or 4. In some cases, the antibody or fragment is an antibody or fragment having 75% or more, 80% or more, 90% or more, 95% or more, or 99% or more homology to such an antibody or fragment.

In other embodiments, the inhibitor of LOXL2 is a nucleic acid. In some aspects, the nucleic acid is selected from the group consisting of a siRNA, a shRNA, a ribozyme and a triplex-forming oligonucleotide. In some aspects, the nucleic acid is a siRNA. In some examples, the siRNA has a sequence selected from the group consisting of SEQ ID NOs: 14 and 15. In some aspects, the nucleic acid is a shRNA. In some examples, the shRNA has a sequence selected from the group consisting of SEQ ID NOs: 16 and 17.

### BRIEF DESCRIPTION OF THE DRAWINGS

FIG. 1, panels A and B, show the effect of a LOXL2 targeted shRNA on properties of human umbilical vein endothelial cells (HUVEC). FIG. 1A shows the migratory ability of cells expressing a shRNA targeted to LOXL2 (shLOXL2) and cells expressing a control shRNA that is not homologous to LOXL2 sequences (shC). FIG. 1B shows the number of branchpoints detected in a Matrigel tube formation assay formed by cells expressing a shRNA targeted to LOXL2 (shLOXL2) and cells expressing a control shRNA that is not homologous to LOXL2 sequences (shC).

FIG. 2, panels A and B, show the effect of an anti-LOXL2 antibody on properties of human umbilical vein endothelial cells (HUVEC). FIG. 2A shows the migratory ability of cells exposed to the anti-LOXL2 antibody AB0023 and cells exposed to a control antibody. FIG. 2B shows the number of branchpoints detected in a Matrigel tube formation assay formed by cells exposed to the anti-LOXL2 antibody AB0023 and cells exposed to a control antibody.

FIG. 3 shows levels of CD31 (an endothelial marker) in sections from Matrigel plugs that had been implanted in mice treated either with an anti-LOXL2 antibody (AB0023) or PBST (Vehicle).

FIG. 4, panels A and B, show levels of angiogenic markers in sections from Matrigel plugs that had been implanted in mice treated either with an anti-LOXL2 antibody (AB0023) or PBST (Vehicle). FIG. 4A shows levels of CD34 (a marker for endothelial cells) and FIG. 4B shows levels of NG2 (a pericyte marker).

FIG. 5 shows a comparison of CD34 levels in sections from Matrigel plugs that had been implanted in mice treated with an anti-LOXL2 antibody (AB0023), a control antibody not directed to LOXL2 (AC-1) or PBST (Vehicle).

FIG. 6, panels A and B, show number of cells expressing certain angiogenic markers, determined by flow cytometry, in Matrigel plugs that had been implanted in mice treated either with an anti-LOXL2 antibody (AB0023) or PBST (Vehicle). FIG. 6A shows numbers of cells expressing CD31 (an endothelial marker). FIG. 6B shows numbers of cells that express VEGF receptor-2 (VEGFR2) and do not express CD45.

FIG. 7 shows measurements of average tumor volume in mice with tumors resulting from transplantation of SKOV3 tumor cells. After tumor volume reached 100 mm³, animals were administered vehicle (open circles), anti-LOXL2 antibody AB0023 (filled circles), taxol (open triangles) or a combination of AB0023 and taxol (closed inverted triangles).

FIG. 8 shows measurements of the relative average area of CD31 immunoreactivity in frozen sections of tumors from animals that had been treated with the anti-LOXL2 antibody AB0023 (squares) and from control animals (circles). Two representative sections were examined from five vehicle or five AB0023-treated tumors. p<0.01.

FIG. 9 shows measurements the average area of carbonic anhydrase IX (CA9) immunoreactivity in frozen sections of tumor tissue from animals treated with (from left to right) vehicle, AB0023, taxol, and AB0023+taxol (AB23/Taxol). Three representative 10× fields from a minimum of three tumors per condition were assessed for this analysis. p<0.05

FIG. 10 shows the average number of perfused tumor vessels, determined by coincidence of Hoechst 33342 and CD31 staining, in section of tumor tissue from control mice (circles) and AB0023-treated mice (squares). Representative sections were examined from four AC-1-treated and four AB0023-treated tumors. p<0.05.

FIG. 11 shows measurements of the area of NG2⁺ cells associated with or overlapping with CD31⁺ cells, expressed as a fraction of total CD31⁺ cells in the section. Animals bearing experimental tumors as described in Example 4 were treated with the anti-LOXL2 antibody AB0023 (right, squares) or with vehicle (left, circles). Two representative sections were examined from five vehicle and five AB0023-treated tumors. p<0.01.

### DETAILED DESCRIPTION

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N.J., 2000; and the series “Methods in Enzymology,” Academic Press, San Diego, Calif.

### DEFINITIONS

“Angiogenesis” refers to the process by which new blood vessels sprout from existing vessels.

“Vasculogenesis” refers to the process by which new blood vessels are formed de novo, e.g., by recruitment of endothelial cells (e.g., from bone marrow).

“Normalization of vasculature” or “vascular normalization” refers to conversion of a dense network of leaky, tortuous, disorganized vessels (e.g., tumor vessels) to a more organized network of generally fewer vessels (i.e., a less dense network) that are less permeable, less dilated and less tortuous. Vascular normalization is also characterized by increased association of pericytes with the endothelial cells lining the walls of the vessels, formation of a more normal basement membrane (e.g., having a more physiological thickness) and closer association of vessels with the basement membrane. Normalization of vasculature can also involve pruning of immature vessels, along with increased integrity and stability of the remaining vasculature.

The results of vascular normalization include decreased interstitial fluid pressure in a tumor and generation of a hydrostatic pressure gradient across the walls of the vessels. Normalization of tumor vasculature is also characterized by increased perfusion of the tumor, reduced tumor hypoxia, and reduced leakiness of vessels. For all of these reasons, tumors with normalized vasculature can be more easily and more deeply penetrated by anti-neoplastic agents (e.g., chemotherapeutic drugs or biologics) and are more susceptible to therapies that are enhanced by the presence of oxygen, such as, e.g., radiation. See, e.g., Jain (2005) Science 307:58-62 and Tong et al. (2004) 64:3731-3736.

“Pericytes” (also known as “perivascular cells” or “adventitial cells”) are cells that lie in close association to endothelial cells, distal to the vessel lumen, in normal vasculature. Pericytes have diverse functions, including maintenance of vascular integrity and regulation of blood flow, and also play a role in angiogenesis. In addition, pericytes are pluripotent mesenchymal cells that can serve as progenitors of, inter alia, fibroblasts and smooth muscle cells (e.g., in vessel walls).

“Hypoxia” refers to a lower-than-physiological oxygen tension in a cell or tissue. Normal physiological oxygen tension, referred to as normoxia, can be between approximately 20-40 mmHg; but any value of oxygen tension below normal physiological oxygen tension for a given condition of a given tissue is considered hypoxic. Hypoxia induces the expression of transcription factors, such as HIF-1α, and induces expression of additional proteins such as, for example, carbonic anhydrase IX (CA9). Areas of hypoxia often surround necrotic regions of tumors.

“Perfusion” refers to the ability of blood to access a tissue or tumor via a vascular network. Higher perfusion is correlated with more uniform access to all regions of the tissue or tumor.

An “inhibitor of LOXL2” or “LOXL2 inhibitor” can be any type of molecule, including but not limited to a low molecular weight organic compound (I.e., “small molecule”), nucleic acid (e.g., triplex-forming oligonucleotide, antisense RNA, ribozyme, shRNA, siRNA, micro RNA), polypeptide (e.g., antibody, peptide mimetic), polysaccharide, or glycoprotein. Means for the identification of LOXL2 inhibitors, and various exemplary inhibitors of LOXL2, are disclosed in the present specification infra.

Inhibition of LOXL2 activity can be accomplished by any means known in the art, including, but not limited to, mutagenesis of a gene encoding LOXL2 and/or of sequences regulating its expression; inhibition of transcription of a gene encoding LOXL2; alteration of a mRNA encoding LOXL2; inhibition of translation of a mRNA encoding LOXL2; and/or inhibition of the enzymatic activity of LOXL2.

Inhibition of the enzymatic activity of LOXL2 can be achieved using molecules that bind to LOXL2; e.g., low molecular weight organic compounds (I.e. “small molecules”) and/or antibodies. Other exemplary inhibitors include nucleic acids, polypeptides and peptidomimetics. In certain embodiments, non-competitive inhibitors of LOXL2 are used. Inhibition of LOXL2 activity can also be achieved by inhibiting the activity or production of enzymes that are involved in the synthesis of LOXL2 or by inhibiting enzymes involved in the synthesis of a substrate of LOXL2 or a precursor of said substrate.

An “anti-neoplastic agent,” as disclosed herein, is any molecule that is used in the treatment of cancer, for example, a low molecular weight organic compound (I.e., a “small molecule” chemotherapeutic drug), a protein (e.g., a therapeutic antibody) or a nucleic acid (e.g., an antisense RNA, ribozyme, shRNA or siRNA). Radiation is also considered to be an anti-neoplastic agent. Chemotherapeutics include low-molecular weight organic compounds (I.e., “small molecules”) and therapeutic biological macromolecules (e.g., antibodies). Further description and examples of anti-neoplastic agents are provided in the specification infra.

Inhibition of LOXL2 and Vascular Normalization

LOXL2 is a member of a family of enzymes (lysyl oxidase-type enzymes) that crosslinks collagen and elastin to form collagen fibers and elastin fibers in the extracellular matrix. LOXL2 is expressed by endothelial cells and has been described as having a role in angiogenesis. See, e.g., US 2010/0119515 (May 13, 2010). However, the qualitative effects of LOXL2 activity on vasculature have not heretofore been examined.

It is disclosed herein that systemic inhibition of LOXL2 activity in tumor-bearing animals resulted in a quantitative reduction in vessel number, and in qualitative changes in tumor vasculature. In particular, inhibition of LOXL2 resulted in conversion of disorganized, leaky tumor vasculature to a more normal, less permeable physiological state. Thus, treatment of tumor-bearing animals with an inhibitor of LOXL2 decreased tumor hypoxia, improved perfusion and increased the association of pericytes with tumor-associated vessels, without an attendant increase in pericyte number. The more efficient tumor vasculature induced by LOXL2 inhibition enabled enhanced delivery of co-administered chemotherapeutic agents, and rendered tumors more sensitive to those agents.

In certain model systems characterized by highly vascularized tumors, treatment of tumor-bearing animals with a LOXL2 inhibitor did not inhibit tumor growth; however, it did potentiate the growth-inhibitory effect of the chemotherapeutic drug taxol. Thus, inhibition of LOXL2 also worked synergistically with a chemotherapeutic to block tumor growth. See Example 5, infra.

Because LOXL2 is expressed at low levels, if at all, in most normal tissues, systemic inhibition of LOXL2 has no obvious detrimental effects on normal vasculature or on the cardiovascular system. Thus, the use of LOXL2 inhibitors as a therapeutic modality results in significant efficacy accompanied by minimal side effects.

Lysyl Oxidase-Type Enzymes

As used herein, the term “lysyl oxidase-type enzyme” refers to a member of a family of proteins that, inter alia, catalyzes oxidative deamination of 8-amino groups of lysine and hydroxylysine residues, resulting in conversion of peptidyl lysine to peptidyl-α-aminoadipic-δ-semialdehyde (allysine) and the release of stoichiometric quantities of ammonia and hydrogen peroxide:

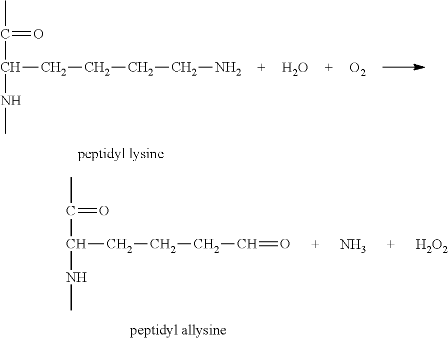

This reaction most often occurs extracellularly, on lysine residues in collagen and elastin. The aldehyde residues of allysine are reactive and can spontaneously condense with other allysine and lysine residues, resulting in crosslinking of collagen molecules to form collagen fibrils.

Lysyl oxidase-type enzymes have been purified from chicken, rat, mouse, bovines and humans. All lysyl oxidase-type enzymes contain a common catalytic domain, approximately 205 amino acids in length, located in the carboxy-terminal portion of the protein and containing the active site of the enzyme. The active site contains a copper-binding site which includes a conserved amino acid sequence containing four histidine residues which coordinate a Cu(II) atom. The active site also contains a lysyltyrosyl quinone (LTQ) cofactor, formed by intramolecular covalent linkage between a lysine and a tyrosine residue (corresponding to lys314 and tyr349 in rat lysyl oxidase, and to lys320 and tyr355 in human lysyl oxidase). The sequence surrounding the tyrosine residue that forms the LTQ cofactor is also conserved among lysyl oxidase-type enzymes. The catalytic domain also contains ten conserved cysteine residues, which participate in the formation of five disulfide bonds. The catalytic domain also includes a fibronectin binding domain. Finally, an amino acid sequence similar to a growth factor and cytokine receptor domain, containing four cysteine residues, is present in the catalytic domain. Despite the presence of these conserved regions, the different lysyl oxidase-type enzymes can be distinguished from one another, both within and outside their catalytic domains, by virtue of regions of divergent nucleotide and amino acid sequence.

The first member of this family of enzymes to be isolated and characterized was lysyl oxidase (EC 1.4.3.13); also known as protein-lysine 6-oxidase, protein-L-lysine:oxygen 6-oxidoreductase (deaminating), or LOX. See, e.g., Harris et al., Biochim. Biophys. Acta 341:332-344 (1974); Rayton et al., J. Biol. Chem. 254:621-626 (1979); Stassen, Biophys. Acta 438:49-60 (1976).

Additional lysyl oxidase-type enzymes were subsequently discovered. These proteins have been dubbed “LOX-like,” or “LOXL.” They all contain the common catalytic domain described above and have similar enzymatic activity. Currently, five different lysyl oxidase-type enzymes are known to exist in both humans and mice: LOX and the four LOX related, or LOX-like proteins LOXL1 (also denoted “lysyl oxidase-like,” “LOXL” or “LOL”), LOXL2 (also denoted “LOR-1”), LOXL3 (also denoted “LOR-2”), and LOXL4. Each of the genes encoding the five different lysyl oxidase-type enzymes resides on a different chromosome. See, for example, Molnar et al., Biochim Biophys Acta. 1647:220-24 (2003); Csiszar, Prog. Nucl. Acid Res. 70:1-32 (2001); WO 01/83702 published on Nov. 8, 2001, and U.S. Pat. No. 6,300,092, all of which are incorporated by reference herein. A LOX-like protein termed LOXC, with some similarity to LOXL4 but with a different expression pattern, has been isolated from a murine EC cell line. Ito et al. (2001) J. Biol. Chem. 276:24023-24029. Two lysyl oxidase-type enzymes, DmLOXL-1 and DmLOXL-2, have been isolated from Drosophila.

Although all lysyl oxidase-type enzymes share a common catalytic domain, they also differ from one another, particularly in their amino-terminal regions. The four LOXL proteins have amino-terminal extensions, compared to LOX. Thus, while human preproLOX (i.e., the primary translation product prior to signal sequence cleavage, see below) contains 417 amino acid residues; LOXL1 contains 574, LOXL2 contains 638, LOXL3 contains 753 and LOXL4 contains 756.

Within their amino-terminal regions, LOXL2, LOXL3 and LOXL4 contain four repeats of the scavenger receptor cysteine-rich (SRCR) domain. These domains are not present in LOX or LOXL1. SRCR domains are found in secreted, transmembrane, or extracellular matrix proteins, and are known to mediate ligand binding in a number of secreted and receptor proteins. Hoheneste et al. (1999) Nat. Struct. Biol. 6:228-232; Sasaki et al. (1998) EMBO J. 17:1606-1613. In addition to its SRCR domains, LOXL3 contains a nuclear localization signal in its amino-terminal region. A proline-rich domain appears to be unique to LOXL1. Molnar et al. (2003) Biochim. Biophys. Acta 1647:220-224. The various lysyl oxidase-type enzymes also differ in their glycosylation patterns.

Tissue distribution also differs among the lysyl oxidase-type enzymes. Human LOX mRNA is highly expressed in the heart, placenta, testis, lung, kidney and uterus, but marginally in the brain and liver. mRNA for human LOXL1 is expressed in the placenta, kidney, muscle, heart, lung, and pancreas and, similar to LOX, is expressed at much lower levels in the brain and liver. Kim et al. (1995) J. Biol. Chem. 270:7176-7182. High levels of LOXL2 mRNA are expressed in the uterus, placenta, and other organs, but as with LOX and LOXL1, low levels are expressed in the brain and liver. Jourdan Le-Saux et al. (1999) J. Biol. Chem. 274:12939:12944. LOXL3 mRNA is highly expressed in the testis, spleen, and prostate, moderately expressed in placenta, and not expressed in the liver, whereas high levels of LOXL4 mRNA are observed in the liver. Huang et al. (2001) Matrix Biol. 20:153-157; Maki and Kivirikko (2001) Biochem. J. 355:381-387; Jourdan Le-Saux et al. (2001) Genomics 74:211-218; Asuncion et al. (2001) Matrix Biol. 20:487-491.

The expression and/or involvement of the different lysyl oxidase-type enzymes in diseases also varies. See, for example, Kagan (1994) Pathol. Res. Pract. 190:910-919; Murawaki et al. (1991) Hepatology 14:1167-1173; Siegel et al. (1978) Proc. Natl. Acad. Sci. USA 75:2945-2949; Jourdan Le-Saux et al. (1994) Biochem. Biophys. Res. Comm. 199:587-592; and Kim et al. (1999) J. Cell Biochem. 72:181-188. Lysyl oxidase-type enzymes have also been implicated in a number of cancers, including head and neck cancer, bladder cancer, colon cancer, esophageal cancer and breast cancer. See, for example, Wu et al. (2007) Cancer Res. 67:4123-4129; Gorough et al. (2007) J. Pathol. 212:74-82; Csiszar (2001) Prog. Nucl. Acid Res. 70:1-32 and Kirschmann et al. (2002) Cancer Res. 62:4478-4483.

Thus, although the lysyl oxidase-type enzymes exhibit some overlap in structure and function, each has distinct structure and functions as well. With respect to structure, for example, certain antibodies raised against the catalytic domain of the human LOX protein do not bind to human LOXL2. With respect to function, it has been reported that targeted deletion of LOX appears to be lethal at parturition in mice, whereas LOXL1 deficiency causes no severe developmental phenotype. Hornstra et al. (2003) J. Biol. Chem. 278:14387-14393; Bronson et al. (2005) Neurosci. Lett. 390:118-122.

Although the most widely documented activity of lysyl oxidase-type enzymes is the oxidation of specific lysine residues in collagen and elastin outside of the cell, there is evidence that lysyl oxidase-type enzymes also participate in a number of intracellular processes. For example, there are reports that some lysyl oxidase-type enzymes regulate gene expression. Li et al. (1997) Proc. Natl. Acad. Sci. USA 94:12817-12822; Giampuzzi et al. (2000) J. Biol. Chem. 275:36341-36349. In addition, LOX has been reported to oxidize lysine residues in histone H1. Additional extracellular activities of LOX include the induction of chemotaxis of monocytes, fibroblasts and smooth muscle cells. Lazarus et al. (1995) Matrix Biol. 14:727-731; Nelson et al. (1988) Proc. Soc. Exp. Biol. Med. 188:346-352. Expression of LOX itself is induced by a number of growth factors and steroids such as TGF-13, TNF-α, and interferon. Csiszar (2001) Prog. Nucl. Acid Res. 70:1-32. Recent studies have attributed other roles to LOX in diverse biological functions such as developmental regulation, tumor suppression, cell motility, and cellular senescence.

Examples of lysyl oxidase (LOX) proteins from various sources include enzymes having an amino acid sequence substantially identical to a polypeptide expressed or translated from one of the following sequences: EMBL/GenBank accessions: M94054; AAA59525.1—mRNA; S45875; AAB23549.1—mRNA; S78694; AAB21243.1—mRNA; AF039291; AAD02130.1—mRNA; BC074820; AAH74820.1—mRNA; BC074872; AAH74872.1—mRNA; M84150; AAA59541.1—Genomic DNA. One embodiment of LOX is human lysyl oxidase (hLOX) preproprotein.

Exemplary disclosures of sequences encoding lysyl oxidase-like enzymes are as follows: LOXL1 is encoded by mRNA deposited at GenBank/EMBL BC015090; AAH15090.1; LOXL2 is encoded by mRNA deposited at GenBank/EMBL U89942; LOXL3 is encoded by mRNA deposited at GenBank/EMBL AF282619; AAK51671.1; and LOXL4 is encoded by mRNA deposited at GenBank/EMBL AF338441; AAK71934.1.

The primary translation product of the LOX protein, known as the prepropeptide, contains a signal sequence extending from amino acids 1-21. This signal sequence is released intracellularly by cleavage between Cys21 and Ala22, in both mouse and human LOX, to generate a 46-48 kDa propeptide form of LOX, also referred to herein as the full-length form. The propeptide is N-glycosylated during passage through the Golgi apparatus to yield a 50 kDa protein, then secreted into the extracellular environment. At this stage, the protein is catalytically inactive. A further cleavage, between Gly168 and Asp169 in mouse LOX, and between Gly174 and Asp175 in human LOX, generates the mature, catalytically active, 30-32 kDA enzyme, releasing a 18 kDa propeptide. This final cleavage event is catalyzed by the metalloendoprotease procollagen C-proteinase, also known as bone morphogenetic protein-1 (BMP-1). Interestingly, this enzyme also functions in the processing of LOX's substrate, collagen. The N-glycosyl units are subsequently removed.

Potential signal peptide cleavage sites have been predicted at the amino termini of LOXL1, LOXL2, LOXL3, and LOXL4. The predicted signal cleavage sites are between Gly25 and Gln26 for LOXL1, between Ala25 and Gln26, for LOXL2, between Gly25 and Ser26 for LOXL3 and between Arg23 and Pro24 for LOXL4.

A BMP-1 cleavage site in the LOXL1 protein has been identified between Ser354 and Asp355. Borel et al. (2001) J. Biol. Chem. 276:48944-48949. Potential BMP-1 cleavage sites in other lysyl oxidase-type enzymes have been predicted, based on the consensus sequence for BMP-1 cleavage in procollagens and pro-LOX being at an Ala/Gly-Asp sequence, often followed by an acidic or charged residue. A predicted BMP-1 cleavage site in LOXL3 is located between Gly447 and Asp448; processing at this site may yield a mature peptide of similar size to mature LOX. A potential cleavage site for BMP-1 was also identified within LOXL4, between residues Ala569 and Asp570. Kim et al. (2003) J. Biol. Chem. 278:52071-52074. LOXL2 may also be proteolytically cleaved analogously to the other members of the LOXL family and secreted. Akiri et al. (2003) Cancer Res. 63:1657-1666.

As expected from the existence of a common catalytic domain in the lysyl oxidase-type enzymes, the sequence of the C-terminal 30 kDa region of the proenzyme in which the active site is located is highly conserved (approximately 95%). A more moderate degree of conservation (approximately 60-70%) is observed in the propeptide domain.

For the purposes of the present disclosure, the term “lysyl oxidase-type enzyme” encompasses all five of the lysine oxidizing enzymes discussed above (LOX, LOXL1, LOXL2, LOXL3 and LOXL4); while each of the terms LOX, LOXL1, LOXL2, LOXL3 and LOXL4, when used singly, refer to a single specific protein. Functional fragments are derivatives of LOX, LOXL1, LOXL2, LOXL3 and LOXL4 that substantially retain enzymatic activity; e.g., the ability to catalyze deamination of lysyl residues. Typically, a functional fragment or derivative retains at least 50% of its lysine oxidation activity. In some embodiments, a functional fragment or derivative retains at least 60%, at least 70%, at least 80%, at least 90%, at least 95%, at least 99% or 100% of its lysine oxidation activity.

It is also intended that a functional fragment of a lysyl oxidase-type enzyme (e.g., LOXL2) can include conservative amino acid substitutions (with respect to the native polypeptide sequence) that do not substantially alter catalytic activity. The term “conservative amino acid substitution” refers to grouping of amino acids on the basis of certain common structures and/or properties. With respect to common structures, amino acids can be grouped into those with non-polar side chains (glycine, alanine, valine, leucine, isoleucine, methionine, proline, phenylalanine and tryptophan), those with uncharged polar side chains (serine, threonine, asparagine, glutamine, tyrosine and cysteine) and those with charged polar side chains (lysine, arginine, aspartic acid, glutamic acid and histidine). A group of amino acids containing aromatic side chains includes phenylalanine, tryptophan and tyrosine. Heterocyclic side chains are present in proline, tryptophan and histidine. Within the group of amino acids containing non-polar side chains, those with short hydrocarbon side chains (glycine, alanine, valine, leucine, isoleucine) can be distinguished from those with longer, non-hydrocarbon side chains (methionine, proline, phenylalanine, tryptophan). Within the group of amino acids with charged polar side chains, the acidic amino acids (aspartic acid, glutamic acid) can be distinguished from those with basic side chains (lysine, arginine and histidine).

A functional method for defining common properties of individual amino acids is to analyze the normalized frequencies of amino acid changes between corresponding proteins of homologous organisms (Schulz, G. E. and R. H. Schirmer, Principles of Protein Structure, Springer-Verlag, 1979). According to such analyses, groups of amino acids can be defined in which amino acids within a group are preferentially substituted for one another in homologous proteins, and therefore have similar impact on overall protein structure (Schulz, G. E. and R. H. Schirmer, Principles of Protein Structure, Springer-Verlag, 1979). According to this type of analysis, the following groups of amino acids that can be conservatively substituted for one another can be identified:
(i) amino acids containing a charged group, consisting of Glu, Asp, Lys, Arg and His,
(ii) amino acids containing a positively-charged group, consisting of Lys, Arg and His,
(iii) amino acids containing a negatively-charged group, consisting of Glu and Asp,
(iv) amino acids containing an aromatic group, consisting of Phe, Tyr and Trp,
(v) amino acids containing a nitrogen ring group, consisting of His and Trp,
(vi) amino acids containing a large aliphatic non-polar group, consisting of Val, Leu and Be,
(vii) amino acids containing a slightly-polar group, consisting of Met and Cys,
(viii) amino acids containing a small-residue group, consisting of Ser, Thr, Asp, Asn, Gly, Ala, Glu, Gln and Pro,
(ix) amino acids containing an aliphatic group consisting of Val, Leu, Ile, Met and Cys, and
(x) amino acids containing a hydroxyl group consisting of Ser and Thr.

Thus, as exemplified above, conservative substitutions of amino acids are known to those of skill in this art and can be made generally without altering the biological activity of the resulting molecule. Those of skill in this art also recognize that, in general, single amino acid substitutions in non-essential regions of a polypeptide do not substantially alter biological activity. See, e.g., Watson, et al., “Molecular Biology of the Gene,” 4th Edition, 1987, The Benjamin/Cummings Pub. Co., Menlo Park, Calif., p. 224.

Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site is occupied by a similar amino acid residue (e.g., similar in steric and/or electronic nature or a conservatively-substituted amino acid), then the molecules can be referred to as homologous (similar) at that position. Expression of a percentage of homology/similarity or identity refers to a function of the number of identical or similar amino acids at positions shared by the compared sequences. A sequence which is “unrelated” or “non-homologous” shares less than 40% identity, or less than 25% identity, with a reference sequence. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. A reference amino acid (protein) sequence (e.g., a sequence shown herein) may be used as a “query sequence” to perform a search against public databases to, for example, identify other family members, related sequences or homologues. Such searches can be performed using, for example, the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a reference nucleic acid. BLAST amino acid searches can be performed with the XBLAST program, score=50, wordlength=3 to obtain amino acid sequences homologous to a reference amino acid sequence. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (see, e.g., the world wide web at: ncbi.nlm.nih.gov).

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “substantially identical” means identity between a first amino acid sequence that contains a sufficient or minimum number of amino acid residues that are (i) identical to, or (ii) conservative substitutions of, aligned amino acid residues in a second amino acid sequence such that the first and second amino acid sequences can have a common structural domain and/or common functional activity. For example, amino acid sequences that contain a common structural domain having at least about 90%, 91%, 92%, 93%, 94%, 95%, 96%, 97%, 98% or 99% identity to LOXL2 are termed sufficiently or substantially identical to the LOXL2 polypeptide. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity.

In certain embodiments of the present disclosure, inhibitors of the lysyl oxidase-like-2 (LOXL2) protein, previously identified as lysyl oxidase-related protein-1 (LOR-1) are used. The LOXL2 protein is initially synthesized as a preproenzyme containing, from amino- to carboxy-termini, a signal sequence, a SRCR1 domain, a SRCR2 domain, a SRCR3 domain, a SRCR4 domain and a catalytic domain. Cleavage in the spacer region between the SRCR2 and SRCR3 domains yields a mature protein containing the SRCR3, SRCR4 and catalytic domains.

For additional information regarding lysyl oxidase-type enzymes, see, e.g., Rucker et al. (1998) Am. J. Clin. Nutr. 67:996 S-1002S and Kagan et al. (2003) J. Cell. Biochem 88:660-672. See also co-owned United States patent application publication Nos. 2009/0053224 (Feb. 26, 2009) and 2009/0104201 (Apr. 23, 2009); the disclosures of which are incorporated by reference herein.

Modulators of the Activity of Lysyl Oxidase-Type Enzymes

Modulators of the activity of lysyl oxidase-type enzymes (e.g., LOXL2) include both activators (agonists) and inhibitors (antagonists), and can be selected by using a variety of screening assays. In one embodiment, modulators can be identified by determining if a test compound binds to LOXL2; wherein, if binding has occurred, the compound is a candidate modulator. Optionally, additional tests can be carried out on such a candidate modulator. Alternatively, a candidate compound can be contacted with a lysyl oxidase-type enzyme, and a biological activity of the lysyl oxidase-type enzyme assayed; a compound that alters the biological activity of the lysyl oxidase-type enzyme is a modulator of a lysyl oxidase-type enzyme. Generally, a compound that reduces a biological activity of a lysyl oxidase-type enzyme is an inhibitor of the enzyme.

Other methods of identifying modulators of the activity of lysyl oxidase-type enzymes (e.g., LOXL2) include incubating a candidate compound in a cell culture containing one or more lysyl oxidase-type enzymes and assaying one or more biological activities or characteristics of the cells. Compounds that alter the biological activity or characteristic of the cells in the culture are potential modulators of the activity of a lysyl oxidase-type enzyme. Biological activities that can be assayed include, for example, lysine oxidation, peroxide production, ammonia production, levels of LOXL2, levels of mRNA encoding LOXL2, and/or one or more functions specific to LOXL2 In additional embodiments of the aforementioned assay, in the absence of contact with the candidate compound, the one or more biological activities or cell characteristics are correlated with levels or activity of one or more lysyl oxidase-type enzymes. For example, the biological activity can be a cellular function such as migration, chemotaxis, epithelial-to-mesenchymal transition, or mesenchymal-to-epithelial transition, and the change is detected by comparison with one or more control or reference sample(s). For example, negative control samples can include a culture with decreased levels of a lysyl oxidase-type enzyme to which the candidate compound is added; or a culture with the same amount of lysyl oxidase-type enzyme as the test culture, but without addition of candidate compound. In some embodiments, separate cultures containing different levels of e.g., LOXL2, are contacted with a candidate compound. If a change in biological activity is observed, and if the change is greater in the culture having higher levels of LOXL2, the compound is identified as a modulator of the activity of LOXL2. Determination of whether the compound is an activator or an inhibitor of LOXL2 may be apparent from the phenotype induced by the compound, or may require further assay, such as a test of the effect of the compound on LOXL2 enzymatic activity.

Methods for obtaining lysysl oxidase-type enzymes such as LOXL2, either biochemically or recombinantly, as well as methods for cell culture and enzymatic assay to identify modulators of the activity of lysyl oxidase-type enzymes as described above, are known in the art. For example, the isolated catalytic domain of LOXL2, as disclosed in co-owned International Patent Application Publication No. WO 2011/022667, entitled “Catalytic Domains from Lysyl Oxidase and LOXL2,” can be used to screen for inhibitors of LOXL2.

The enzymatic activity of a lysyl oxidase-type enzyme can be assayed by a number of different methods. For example, lysyl oxidase enzymatic activity can be assessed by detecting and/or quantitating production of hydrogen peroxide, ammonium ion, and/or aldehyde, by assaying lysine oxidation and/or collagen crosslinking, or by measuring cellular invasive capacity, cell adhesion, cell growth or metastatic growth. See, for example, Trackman et al. (1981) Anal. Biochem. 113:336-342; Kagan et al. (1982) Meth. Enzymol. 82A:637-649; Palamakumbura et al. (2002) Anal. Biochem. 300:245-251; Albini et al. (1987) Cancer Res. 47:3239-3245; Kamath et al. (2001) Cancer Res. 61:5933-5940; U.S. Pat. No. 4,997,854 and U.S. patent application publication No. 2004/0248871.

Test compounds include, but are not limited to, small organic compounds (e.g., organic molecules having a molecular weight between about 50 and about 2,500 Da), nucleic acids or proteins, for example. The compound or plurality of compounds can be chemically synthesized or microbiologically produced and/or comprised in, for example, samples, e.g., cell extracts from, e.g., plants, animals or microorganisms. Furthermore, the compound(s) can be known in the art but hitherto not known to be capable of modulating the activity of a lysyl oxidase-type enzyme. The reaction mixture for assaying for a modulator of a lysyl oxidase-type enzyme can be a cell-free extract or can comprise a cell culture or tissue culture. A plurality of compounds can be, e.g., added to a reaction mixture, added to a culture medium, injected into a cell or administered to a transgenic animal. The cell or tissue employed in the assay can be, for example, a bacterial cell, a fungal cell, an insect cell, a vertebrate cell, a mammalian cell, a primate cell, a human cell or can comprise or be obtained from a non-human transgenic animal.

Several methods are known to the person skilled in the art for producing and screening large libraries to identify compounds having specific affinity for a target, such as a lysyl oxidase-type enzyme. These methods include phage display method in which randomized peptides are displayed from phage and screened by affinity chromatography using an immobilized receptor. See, e.g., WO 91/17271, WO 92/01047, and U.S. Pat. No. 5,223,409. In another approach, combinatorial libraries of polymers immobilized on a solid support (e.g., a “chip”) are synthesized using photolithography. See, e.g., U.S. Pat. No. 5,143,854, WO 90/15070 and WO 92/10092. The immobilized polymers are contacted with a labeled receptor (e.g., a lysyl oxidase-type enzyme) and the support is scanned to determine the location of label, to thereby identify polymers binding to the receptor.

The synthesis and screening of peptide libraries on continuous cellulose membrane supports that can be used for identifying binding ligands of a polypeptide of interest (e.g., a lysyl oxidase-type enzyme) is described, for example, in Kramer (1998) Methods Mol. Biol. 87: 25-39. Ligands identified by such an assay are candidate modulators of the protein of interest, and can be selected for further testing. This method can also be used, for example, for determining the binding sites and the recognition motifs in a protein of interest. See, for example Rudiger (1997) EMBO J. 16:1501-1507 and Weiergraber (1996) FEBS Lett. 379:122-126.

WO 98/25146 describes additional methods for screening libraries of complexes for compounds having a desired property, e.g., the capacity to agonize, bind to, or antagonize a polypeptide or its cellular receptor. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with a lysyl oxidase-type enzyme are, for example, in vitro screening with a phage display system, filter binding assays, and “real time” measuring of interaction using, for example, the BIAcore apparatus (Pharmacia).

All these methods can be used in accordance with the present disclosure to identify activators/agonists and inhibitors/antagonists of lysyl oxidase-type enzymes (e.g., LOXL2).

Another approach to the synthesis of modulators of lysyl oxidase-type enzymes is to use mimetic analogs of peptides. Mimetic peptide analogues can be generated by, for example, substituting stereoisomers, i.e. D-amino acids, for naturally-occurring amino acids; see e.g., Tsukida (1997) J. Med. Chem. 40:3534-3541. Furthermore, pro-mimetic components can be incorporated into a peptide to reestablish conformational properties that may be lost upon removal of part of the original polypeptide. See, e.g., Nachman (1995) Regul. Pept. 57:359-370.

Another method for constructing peptide mimetics is to incorporate achiral O-amino acid residues into a peptide, resulting in the substitution of amide bonds by polymethylene units of an aliphatic chain. Banerjee (1996) Biopolymers 39:769-777. Superactive peptidomimetic analogues of small peptide hormones in other systems have been described. Zhang (1996) Biochem. Biophys. Res. Commun. 224:327-331.

Peptide mimetics of a modulator of a lysyl oxidase-type enzyme can also be identified by the synthesis of peptide mimetic combinatorial libraries through successive amide alkylation, followed by testing of the resulting compounds, e.g., for their binding and immunological properties. Methods for the generation and use of peptidomimetic combinatorial libraries have been described. See, for example, Ostresh, (1996) Methods in Enzymology 267:220-234 and Dorner (1996) Bioorg. Med. Chem. 4:709-715. Furthermore, a three-dimensional and/or crystallographic structure of one or more lysyl oxidase-type enzymes can be used for the design of peptide mimetic inhibitors of the activity of one or more lysyl oxidase-type enzymes. Rose (1996) Biochemistry 35:12933-12944; Rutenber (1996) Bioorg. Med. Chem. 4:1545-1558.

The structure-based design and synthesis of low-molecular-weight synthetic molecules that mimic the activity of native biological polypeptides is further described in, e.g., Dowd (1998) Nature Biotechnol. 16:190-195; Kieber-Emmons (1997) Current Opinion Biotechnol. 8:435-441; Moore (1997) Proc. West Pharmacol. Soc. 40:115-119; Mathews (1997) Proc. West Pharmacol. Soc. 40:121-125; and Mukhija (1998) European J. Biochem. 254:433-438.

It is also well known to the person skilled in the art that it is possible to design, synthesize and evaluate mimetics of small organic compounds that, for example, can act as a substrate or ligand of a lysyl oxidase-type enzyme such as, for example, LOXL2. For example, it has been described that D-glucose mimetics of hapalosin exhibited similar efficiency as hapalosin in antagonizing multidrug resistance assistance-associated protein in cytotoxicity. Dinh (1998) J. Med. Chem. 41:981-987.

The structure of LOXL2 can be investigated to guide the selection of modulators such as, for example, small molecules, peptides, peptide mimetics and antibodies. Structural properties of a lysyl oxidase-type enzyme (e.g., LOXL2) can help to identify natural or synthetic molecules that bind to, or function as a ligand, substrate, binding partner or the receptor of, the lysyl oxidase-type enzyme. See, e.g., Engleman (1997) J. Clin. Invest. 99:2284-2292. For example, folding simulations and computer redesign of structural motifs of lysyl oxidase-type enzymes can be performed using appropriate computer programs. Olszewski (1996) Proteins 25:286-299; Hoffman (1995) Comput. Appl. Biosci. 11:675-679. Computer modeling of protein folding can be used for the conformational and energetic analysis of detailed peptide and protein structure. Monge (1995) J. Mol. Biol. 247:995-1012; Renouf (1995) Adv. Exp. Med. Biol. 376:37-45. Appropriate programs can be used for the identification of sites, on lysyl oxidase-type enzymes, that interact with ligands and binding partners, using computer assisted searches for complementary peptide sequences. Fassina (1994) Immunomethods 5:114-120. Additional systems for the design of protein and peptides are described, for example in Berry (1994) Biochem. Soc. Trans. 22:1033-1036; Wodak (1987), Ann. N.Y. Acad. Sci. 501:1-13; and Pabo (1986) Biochemistry 25:5987-5991. The results obtained from the above-described structural analyses can be used for, e.g., the preparation of organic molecules, peptides and peptide mimetics that function as modulators of the activity of one or more lysyl oxidase-type enzymes.

An inhibitor of a lysyl oxidase-type enzyme can be a competitive inhibitor, an uncompetitive inhibitor, a mixed inhibitor or a non-competitive inhibitor. Competitive inhibitors often bear a structural similarity to substrate, usually bind to the active site, and are more effective at lower substrate concentrations. The apparent K_{M} is increased in the presence of a competitive inhibitor. Uncompetitive inhibitors generally bind to the enzyme-substrate complex or to a site that becomes available after substrate is bound at the active site and may distort the active site. Both the apparent K_{M} and the Vₘₐₓ are decreased in the presence of an uncompetitive inhibitor, and substrate concentration has little or no effect on inhibition. Mixed inhibitors are capable of binding both to free enzyme and to the enzyme-substrate complex and thus affect both substrate binding and catalytic activity. Non-competitive inhibition is a special case of mixed inhibition in which the inhibitor binds enzyme and enzyme-substrate complex with equal avidity, and inhibition is not affected by substrate concentration. Non-competitive inhibitors generally bind to enzyme at a region outside the active site. For additional details on enzyme inhibition see, for example, Voet et al. (2008) supra. For enzymes such as the lysyl oxidase-type enzymes, whose natural substrates (e.g., collagen, elastin) are normally present in vast excess in vivo (compared to the concentration of any inhibitor that can be achieved in vivo), noncompetitive inhibitors are advantageous, since inhibition is independent of substrate concentration.

### Antibodies

In certain embodiments, a modulator of a lysyl oxidase-type enzyme is an antibody. In additional embodiments, an antibody is an inhibitor of the activity of a lysyl oxidase-type enzyme. In additional embodiments, an antibody is an inhibitor of the activity of LOXL2.

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Examples of antibody fragments include Fab, Fab′, F(ab′)₂, and Fv fragments; diabodies; linear antibodies (Zapata et al. (1995) Protein Eng. 8(10): 1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three complementarity-determining regions (CDRs) of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fᵥ fragment.

The “F_{ab}” fragment also contains, in addition to heavy and light chain variable regions, the constant domain of the light chain and the first constant domain (CHO of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SHis the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

In some embodiments, an antibody is a humanized antibody or a human antibody. Humanized antibodies include human immununoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different lysyl oxidase-type enzymes, or to two different epitopes on a single lysyl oxidase-type enzyme.

An antibody as disclosed herein can also be an immunoconjugate. Such immunoconjugates comprise an antibody (e.g., to a lysyl oxidase-type enzyme) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope on a particular polypeptide is one that binds to that particular polypeptide or epitope without substantially binding to any other polypeptide or polypeptide epitope. In some embodiments, an antibody of the present disclosure specifically binds to its target with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; in the form of monoclonal antibody, scFv, Fab, or other form of antibody measured at a temperature of about 4° C., 25° C., 37° C. or 42° C.

In certain embodiments, an antibody of the present disclosure binds to one or more processing sites (e.g., sites of proteolytic cleavage) in a lysyl oxidase-type enzyme, thereby effectively blocking processing of the proenzyme or preproenzyme to the catalytically active enzyme, thereby reducing the activity of the lysyl oxidase-type enzyme.

In certain embodiments, an antibody according to the present disclosure binds to human LOXL2, with a greater binding affinity, for example, 10 times, at least 100 times, or even at least 1000 times greater, than its binding affinity to other lysyl oxidase-type enzymes, e.g., LOX, LOXL1, LOXL3, and LOXL4.

In certain embodiments, an antibody according to the present disclosure is a non-competitive inhibitor of the catalytic activity of LOXL2. In certain embodiments, an antibody according to the present disclosure binds outside the catalytic domain of LOXL2. In certain embodiments, an antibody according to the present disclosure binds to the SRCR4 domain of LOXL2. In certain embodiments, an anti-LOXL2 antibody that binds to the SRCR4 domain of LOXL2 and functions as a non-competitive inhibitor is the AB0023 antibody, described in co-owned U.S. Patent Application Publications No. US 2009/0053224 and US 2009/0104201. In certain embodiments, an anti-LOXL2 antibody that binds to the SRCR4 domain of LOXL2 and functions as a non-competitive inhibitor is the AB0024 antibody (a human version of the AB0023 antibody), described in co-owned U.S. Patent Application Publications No. US 2009/0053224 and US 2009/0104201.

Optionally, an antibody according to the present disclosure not only binds to a lysyl oxidase-type enzyme but also reduces or inhibits uptake or internalization of the lysyl oxidase-type enzyme, e.g., via integrin beta 1 or other cellular receptors or proteins. Such an antibody could, for example, bind to extracellular matrix proteins, cellular receptors, and/or integrins.

Exemplary antibodies that recognize lysyl oxidase-type enzymes, and additional disclosure relating to antibodies to lysyl oxidase-type enzymes, is provided in co-owned U.S. Patent Application Publications No. US 2009/0053224 and US 2009/0104201, the disclosures of which are incorporated by reference for the purposes of describing antibodies to lysyl oxidase-type enzymes, their manufacture, and their use.

Anti-LOXL2 Antibodies

A mouse monoclonal antibody directed against LOXL2 has been described in co-owned United States Patent Application Publication No. US 2009/0053224 (Feb. 26, 2009). This antibody is designated AB0023. The sequence of the signal peptide and the variable region of its heavy chain (with the CDRs underlined) is as follows: MEWSRVFIFLLSVTAGVH SQVQLQQSGAELVRPGTSVKVSCKASGYAFTYYLIEWVKQRPGQGLEWIGVINPGSGGTNYNEK FKGKATLTADKSSSTAYMQLSSLTSDDSAVYFCARNWMNFDYWGQGTTLTVSS (SEQ ID NO:1). Additional heavy chain variable region amino acid sequences having 75% or more, 80% or more, 90% or more, 95% or more, or 99% or more homology to SEQ ID NO:1 are also provided.

The sequence of the signal peptide and the variable region of the light chain of the AB0023 antibody (with the CDRs underlined) is: MRCLAEFLGLLVLWIPGAIGDIVMTQAAP SVSVTPGESVSISCRSSKSLLHSNGNTYLYWFLQRPGQSPQFLIYRMSNLASGVPDRFSGSGSG TAFTLRISRVEAEDVGVYYCMQHLEYPYTFGGGTKLEIK (SEQ ID NO:2). Additional light chain variable region amino acid sequences having 75% or more, 80% or more, 90% or more, 95% or more, or 99% or more homology to SEQ ID NO:2 are also provided.

Humanized versions of the above-mentioned anti-LOXL2 monoclonal antibody have been described in co-owned United States Patent Application Publication No. US 2009/0053224 (Feb. 26, 2009). An exemplary humanized antibody is designated AB0024 or GS-6624. An exemplary sequence of the variable region of its heavy chain (with the CDRs underlined) is as follows: QVQLVQSGAEVKKPGASVKVSCKASGYAFTYYLIEWVRQ APGQGLEWIGVINPGSGGTNYNEKFKGRATITADKSTSTAYMELSSLRSEDTAVYFCARNWMNF DYWGQGTTVTVSS (SEQ ID NO:3). Additional heavy chain variable region amino acid sequences having 75% or more, 80% or more, 90% or more, 95% or more, or 99% or more homology to SEQ ID NO:3 are also provided.

An exemplary sequence of the variable region of the light chain of the AB0024 (GS-6624) antibody (with the CDRs underlined) is: DIVMTQTPLSLSVTPGQPASISCRSSYYCMQHLEYPYTFGGGTKVEIK (SEQ ID NO:4). Additional light chain variable region amino acid sequences having 75% or more, 80% or more, 90% or more, 95% or more, or 99% or more homology to SEQ ID NO:4 are also provided.

Additional anti-LOXL2 antibody sequences, including additional humanized variants of the variable regions, framework region amino acid sequences and the amino acid sequences of the complementarity-determining regions, are disclosed in co-owned United States Patent Application Publication No. US 2009/0053224 (Feb. 26, 2009), the disclosure of which is incorporated by reference in its entirety herein for the purpose of providing the amino acid sequences of various anti-LOXL2 antibodies.

An exemplary nucleotide sequence encoding the AB0023 heavy chain is as follows: ATGGAATGGAGCAGAGTCTTTATCTTTCTCCTATCAGTAACTGCAGGTGTTCACTCCC AGGTCCAGTTGCAGCAGTCTGGAGCTGAGCTGGTAAGGCCTGGGACTTCAGTGAAGGTGTCCTG CAAGGCTTCTGGATACGCCTTCACTTATTACTTGATAGAGTGGGTAAAGCAGAGGCCTGGACAG GGCCTTGAGTGGATTGGGGTGATTAATCCTGGAAGTGGTGGTACTAACTACAATGAGAAGTTCA AGGGCAAGGCAACACTGACTGCAGACAAATCCTCCAGCACTGCCTACATGCAGCTCAGCAGCCT GACATCTGATGACTCTGCGGTCTATTTCTGTGCAAGGAACTGGATGAACTTTGACTACTGGGGC CAAGGCACCACTCTCACAGTCTCCTCA (SEQ ID NO:5).

Sequences encoding the signal peptide and the three complementarity-determining regions (CDRs) are underlined.

An exemplary nucleotide sequence encoding the AB0023 light chain is as follows: ATGAGGTGCCTAGCTGAGTTCCTGGGGCTGCTTGTGCTCTGGATCCCTGGAGCCATTGGGGATA TTGTGATGACTCAGGCTGCACCCTCTGTATCTGTCACTCCTGGAGAGTCAGTATCCATCTCCTG CAGGTCTAGTAAGAGTCTCCTGCATAGTAATGGCAACACTTACTTGTATTGGTTCCTGCAGAGG CCAGGCCAGTCTCCTCAGTTCCTGATATATCGGATGTCCAACCTTGCCTCAGGAGTCCCAGACA GGTTCAGTGGCAGTGGGTCAGGAACTGCTTTCACACTGAGAATCAGTAGAGTGGAGGCTGAGGA TGTGGGTGTTTATTACTGTATGCAACATCTAGAATATCCTTACACGTTCGGAGGGGGGACCAAG CTGGAAATAAAA (SEQ ID NO:6).

Sequences encoding the signal peptide and the three complementarity-determining regions (CDRs) are underlined.

An exemplary nucleotide sequence encoding a humanized heavy chain (variant VH1) ^{_{as follows: CAGGTGCAGCTGGTGCAGTCTGGGGCTGAGCTGAAGAAGCCTGGGGCCT CAGTGAAGGTCTCCTGCAAGGCTTCTGGATACGCCTTCACTTATTACTTGATAGAGTGGGTGAA ACAGGCCCCTGGACAAGGGCTTGAGTGGATCGGAGTGATTAATCCTGGAAGTGGTGGTACTAAC TACAATGAGAAGTTCAAGGGCAGAGCCACGCTCACCGCGGACAAATCCACGAGCACAGCCTACA TGGAGCTGAGCAGCCTGAGATCTGAGGACTCCGCCGTGTATTTCTGTGCGAGAAACTGGATGAA CTTTGACTACTGGGGGCAAGGGACCACGGTCACCGTCTCCTCA (SEQ ID NO:}}7).

Sequences encoding the three complementarity-determining regions (CDRs) are underlined.

Another exemplary nucleotide sequence encoding a humanized heavy chain (variant VH2) ^{_{as follows: CAGGTGCAGCTGGTGCAGTCTGGGGCTGAGgTGAAGAAGCCTGGG GCCTCAGTGAAGGTCTCCTGCAAGGCTTCTGGATACGCCTTCACTTATTACTTGATAGAGTGGG TGAgACAGGCCCCTGGACAAGGGCTTGAGTGGATCGGAGTGATTAATCCTGGAAGTGGTGGTAC TAACTACAATGAGAAGTTCAAGGGCAGAGCCACGCTCACCGCGGACAAATCCACGAGCACAGCC TACATGGAGCTGAGCAGCCTGAGATCTGAGGACaCCGCCGTGTATTTCTGTGCGAGAAACTGGA TGAACTTTGACTACTGGGGGCAAGGGACCACGGTCACCGTCTCCTCA (SEQ ID NO:}}8).

Sequences encoding the three complementarity-determining regions (CDRs) are underlined.

Another exemplary nucleotide sequence encoding a humanized heavy chain (variant VH3) ^{_{as follows: CAGGTGCAGCTGGTGCAGTCTGGGGCTGAGgTGAAGAAGCCTGGG GCCTCAGTGAAGGTCTCCTGCAAGGCTTCTGGATACGCCTTCACTTATTACTTGATAGAGTGGG TGAgACAGGCCCCTGGACAAGGGCTTGAGTGGATCGGAGTGATTAATCCTGGAAGTGGTGGTAC TAACTACAATGAGAAGTTCAAGGGCAGAGCCACGaTCACCGCGGACAAATCCACGAGCACAGCC TACATGGAGCTGAGCAGCCTGAGATCTGAGGACaCCGCCGTGTATTTCTGTGCGAGAAACTGGA TGAACTTTGACTACTGGGGGCAAGGGACCACGGTCACCGTCTCCTCA (SEQ ID NO:}}9).

Sequences encoding the three complementarity-determining regions (CDRs) are underlined.

Another exemplary nucleotide sequence encoding a humanized heavy chain (variant VH4) ^{_{as follows: CAGGTGCAGCTGGTGCAGTCTGGGGCTGAGgTGAAGAAGCCTGG GGCCTCAGTGAAGGTCTCCTGCAAGGCTTCTGGATACGCCTTCACTTATTACTTGATAGAGTGG GTGAgACAGGCCCCTGGACAAGGGCTTGAGTGGATCGGAGTGATTAATCCTGGAAGTGGTGGTA CTAACTACAATGAGAAGTTCAAGGGCAGAGtCACGaTCACCGCGGACAAATCCACGAGCACAGC CTACATGGAGCTGAGCAGCCTGAGATCTGAGGACaCCGCCGTGTATTaCTGTGCGAGAAACTGG ATGAACTTTGACTACTGGGGGCAAGGGACCACGGTCACCGTCTCCTCA (SEQ ID NO:}}10).

Sequences encoding the three complementarity-determining regions (CDRs) are underlined.

An exemplary nucleotide sequence encoding a humanized light chain (variant Vk1) is as follows: GATATTGTGATGACCCAGACTCCACTCTCTCTGTCCGTCACCCCTGGACA GCCGGCCTCCATCTCCTGCAGGTCTAGTAAGAGTCTCCTGCATAGTAATGGCAACA CTTACTTGTATTGGTTCCTGCAGAAGCCAGGGCAGTCTCCACAGTTCCTGATCTATC GGATGTCCAACCTTGCCTCAGGAGTGCCAGATAGGTTCAGTGGCAGCGGGTCAGGG ACAGCCTTCACACTGAAAATCAGCCGGGTGGAGGCTGAGGATGTTGGGGTTTATTA CTGCATGCAACATCTAGAATATCCTTACACCTTCGGCGGAGGGACCAAGGTGGAGA TCAAA (SEQ ID NO:11).

Sequences encoding the three complementarity-determining regions (CDRs) are underlined.

Another exemplary nucleotide sequence encoding a humanized light chain (variant Vk2) ^{_{as follows: GATATTGTGATGACCCAGACTCCACTCTCTCTGTCCGTCACCCCT GGACAGCCGGCCTCCATCTCCTGCAGGTCTAGTAAGAGTCTCCTGCATAGTAATGG CAACACTTACTTGTATTGGTTCCTGCAGAAGCCAGGGCAGTCTCCACAGTTCCTGAT CTATCGGATGTCCAACCTTGCCTCAGGAGTGCCAGATAGGTTCAGTGGCAGCGGGT CAGGGACAGaCTTCACACTGAAAATCAGCCGGGTGGAGGCTGAGGATGTTGGGGTT TATTACTGCATGCAACATCTAGAATATCCTTACACCTTCGGCGGAGGGACCAAGGT GGAGATCAAA (SEQ ID NO:}}12).

Sequences encoding the three complementarity-determining regions (CDRs) are underlined.

Another exemplary nucleotide sequence encoding a humanized light chain (variant Vk3) ^{_{as follows: GATATTGTGATGACCCAGACTCCACTCTCTCTGTCCGTCACCCCT GGACAGCCGGCCTCCATCTCCTGCAGGTCTAGTAAGAGTCTCCTGCATAGTAATGG CAACACTTACTTGTATTGGTaCCTGCAGAAGCCAGGGCAGTCTCCACAGTTCCTGAT CTATCGGATGTCCAACCTTGCCTCAGGAGTGCCAGATAGGTTCAGTGGCAGCGGGT CAGGGACAGaCTTCACACTGAAAATCAGCCGGGTGGAGGCTGAGGATGTTGGGGTT TATTACTGCATGCAACATCTAGAATATCCTTACACCTTCGGCGGAGGGACCAAGGT GGAGATCAAA (SEQ ID NO:}}13).

Sequences encoding the three complementarity-determining regions (CDRs) are underlined.

Polynucleotides for Modulating Expression of Lysyl Oxidase-Type Enzymes

Antisense

Modulation (e.g., inhibition) of a lysyl oxidase-type enzyme can be effected by down-regulating expression of the lysyl oxidase enzyme at either the transcriptional or translational level. One such method of modulation involves the use of antisense oligo- or polynucleotides capable of sequence-specific binding with a mRNA transcript encoding a lysyl oxidase-type enzyme.

Binding of an antisense oligonucleotide (or antisense oligonucleotide analogue) to a target mRNA molecule can lead to the enzymatic cleavage of the hybrid by intracellular RNase H. In certain cases, formation of an antisense RNA-mRNA hybrid can interfere with correct splicing. In both cases, the number of intact, functional target mRNAs, suitable for translation, is reduced or eliminated. In other cases, binding of an antisense oligonucleotide or oligonucleotide analogue to a target mRNA can prevent (e.g., by steric hindrance) ribosome binding, thereby preventing translation of the mRNA.

Antisense oligonucleotides can comprise any type of nucleotide subunit, e.g., they can be DNA, RNA, analogues such as peptide nucleic acids (PNA), or mixtures of the preceding. RNA oligonucleotides form a more stable duplex with a target mRNA molecule, but the unhybridized oligonucleotides are less stable intracellularly than other types of oligonucleotides and oligonucleotide analogues. This can be counteracted by expressing RNA oligonucleotides inside a cell using vectors designed for this purpose. This approach may be used, for example, when attempting to target a mRNA that encodes an abundant and long-lived protein.

Additional considerations can be taken into account when designing antisense oligonucleotides, including: (i) sufficient specificity in binding to the target sequence; (ii) solubility; (iii) stability against intra- and extracellular nucleases; (iv) ability to penetrate the cell membrane; and (v) when used to treat an organism, low toxicity.

Algorithms for identifying oligonucleotide sequences with the highest predicted binding affinity for their target mRNA, based on a thermodynamic cycle that accounts for the energy of structural alterations in both the target mRNA and the oligonucleotide, are available. For example, Walton et al. (1999) Biotechnol. Bioeng. 65:1-9 used such a method to design antisense oligonucleotides directed to rabbit β-globin (RBG) and mouse tumor necrosis factor-α (TNF α) transcripts. The same research group has also reported that the antisense activity of rationally selected oligonucleotides against three model target mRNAs (human lactate dehydrogenase A and B and rat gp130) in cell culture proved effective in almost all cases. This included tests against three different targets in two cell types using oligonucleotides made by both phosphodiester and phosphorothioate chemistries.

In addition, several approaches for designing and predicting efficiency of specific oligonucleotides using an in vitro system are available. See, e.g., Matveeva et al. (1998) Nature Biotechnology 16:1374-1375.

An antisense oligonucleotide according to the present disclosure includes a polynucleotide or a polynucleotide analogue of at least 10 nucleotides, for example, between 10 and 15, between 15 and 20, at least 17, at least 18, at least 19, at least 20, at least 22, at least 25, at least 30, or even at least 40 nucleotides. Such a polynucleotide or polynucleotide analogue is able to anneal or hybridize (i.e., form a double-stranded structure on the basis of base complementarity) in vivo, under physiological conditions, with a mRNA encoding a lysyl oxidase-type enzyme, e.g., LOXL2.

Antisense oligonucleotides according to the present disclosure can be expressed from a nucleic acid construct administered to a cell or tissue. Optionally, expression of the antisense sequences is controlled by an inducible promoter, such that expression of antisense sequences can be switched on and off in a cell or tissue. Alternatively antisense oligonucleotides can be chemically synthesized and administered directly to a cell or tissue, as part of, for example, a pharmaceutical composition.

Antisense technology has led to the generation of highly accurate antisense design algorithms and a wide variety of oligonucleotide delivery systems, thereby enabling those of ordinary skill in the art to design and implement antisense approaches suitable for downregulating expression of known sequences. For additional information relating to antisense technology, see, for example, Lichtenstein et al., “Antisense Technology: A Practical Approach,” Oxford University Press, 1998.

Small RNA and RNAi

Another method for inhibition of the activity of a lysyl oxidase-type enzyme is RNA interference (RNAi), an approach which utilizes double-stranded small interfering RNA (siRNA) molecules that are homologous to a target mRNA and lead to its degradation. Carthew (2001) Curr. Opin. Cell. Biol. 13:244-248.

RNA interference is typically a two-step process. In the first step, which is termed as the initiation step, input dsRNA is digested into 21-23 nucleotide (nt) small interfering RNAs (siRNAs), probably by the action of Dicer, a member of the RNase III family of double-strand-specific ribonucleases, which cleaves double-stranded RNA in an ATP-dependent manner. Input RNA can be delivered, e.g., directly or via a transgene or a virus. Successive cleavage events degrade the RNA to 19-21 bp duplexes (siRNA), each with 2-nucleotide 3′ overhangs. Hutvagner et al. (2002) Curr. Opin. Genet. Dev. 12:225-232; Bernstein (2001) Nature 409:363-366.

In the second, effector step, siRNA duplexes bind to a nuclease complex to form the RNA-induced silencing complex (RISC). An ATP-dependent unwinding of the siRNA duplex is required for activation of the RISC. The active RISC (containing a single siRNA and an RNase) then targets the homologous transcript by base-pairing interactions and typically cleaves the mRNA into fragments of approximately 12 nucleotides, starting from the 3′ terminus of the siRNA. Hutvagner et al., supra; Hammond et al. (2001) Nat. Rev. Gen. 2:110-119; Sharp (2001) Genes. Dev. 15:485-490.

RNAi and associated methods are also described in Tuschl (2001) Chem. Biochem. 2:239-245; Cullen (2002) Nat. Immunol. 3:597-599; and Brantl (2002) Biochem. Biophys. Acta. 1575:15-25.

An exemplary strategy for synthesis of RNAi molecules suitable for use with the present disclosure, as inhibitors of the activity of a lysyl oxidase-type enzyme, is to scan the appropriate mRNA sequence downstream of the start codon for AA dinucleotide sequences. Each AA, plus the downstream (i.e., 3′ adjacent) 19 nucleotides, is recorded as a potential siRNA target site. Target sites in coding regions are preferred, since proteins that bind in untranslated regions (UTRs) of a mRNA, and/or translation initiation complexes, may interfere with binding of the siRNA endonuclease complex. Tuschl (2001) supra. It will be appreciated though, that siRNAs directed at untranslated regions can also be effective, as has been demonstrated in the case wherein siRNA directed at the 5′ UTR of the GAPDH gene mediated about 90% decrease in cellular GAPDH mRNA and completely abolished protein level (www.ambion.com/techlib/tn/91/912.html). Once a set of potential target sites is obtained, as described above, the sequences of the potential targets are compared to an appropriate genomic database (e.g., human, mouse, rat etc.) using a sequence alignment software, (such as the BLAST software available from NCBI located on the World Wide Web at ncbi.nlm.nih.gov/BLAST/). Potential target sites that exhibit significant homology to other coding sequences are rejected.

Qualifying target sequences are selected as templates for siRNA synthesis. Selected sequences can include those with low G/C content as these have been shown to be more effective in mediating gene silencing, compared to those with G/C content higher than 55%. Several target sites can be selected along the length of the target gene for evaluation. For better evaluation of the selected siRNAs, a negative control is used in conjunction. Negative control siRNA can include a sequence with the same nucleotide composition as a test siRNA, but lacking significant homology to the genome. Thus, for example, a scrambled nucleotide sequence of the siRNA may be used, provided it does not display any significant homology to any other gene.

The siRNA molecules of the present disclosure can be transcribed from expression vectors which can facilitate stable expression of the siRNA transcripts once introduced into a host cell. These vectors are engineered to express small hairpin RNAs (shRNAs), which are processed in vivo into siRNA molecules capable of carrying out gene-specific silencing. See, for example, Brummelkamp et al. (2002) Science 296:550-553; Paddison et al (2002) Genes Dev. 16:948-958; Paul et al. (2002) Nature Biotech. 20:505-508; Yu et al. (2002) Proc. Natl. Acad. Sci. USA 99:6047-6052.

Small hairpin RNAs (shRNAs) are single-stranded polynucleotides that form a double-stranded, hairpin loop structure. The double-stranded region is formed from a first sequence that is hybridizable to a target sequence, such as a polynucleotide encoding a lysyl oxidase-type enzyme (e.g., a LOXL2 mRNA) and a second sequence that is complementary to the first sequence. The first and second sequences form a double stranded region; while the un-base-paired linker nucleotides that lie between the first and second sequences form a hairpin loop structure. The double-stranded region (stem) of the shRNA can comprise a restriction endonuclease recognition site.

A shRNA molecule can have optional nucleotide overhangs, such as 2-bp overhangs, for example, 3′ UU-overhangs. While there may be variation, stem length typically ranges from approximately 15 to 49, approximately 15 to 35, approximately 19 to 35, approximately 21 to 31 bp, or approximately 21 to 29 bp, and the size of the loop can range from approximately 4 to 30 bp, for example, about 4 to 23 bp.

For expression of shRNAs within cells, plasmid vectors can be employed that contain a promoter (e.g., the RNA Polymerase III H1-RNA promoter or the U6 RNA promoter), a cloning site for insertion of sequences encoding the shRNA, and a transcription termination signal (e.g., a stretch of 4-5 adenine-thymidine base pairs). Polymerase III promoters generally have well-defined transcriptional initiation and termination sites, and their transcripts lack poly(A) tails. The termination signal for these promoters is defined by the polythymidine tract, and the transcript is typically cleaved after the second encoded uridine. Cleavage at this position generates a 3′ UU overhang in the expressed shRNA, which is similar to the 3′ overhangs of synthetic siRNAs. Additional methods for expressing shRNA in mammalian cells are described in the references cited above.

An example of a suitable shRNA expression vector is pSUPER™ (Oligoengine, Inc., Seattle, Wash.), which includes the polymerase-III H1-RNA gene promoter with a well defined transcriptional startsite and a termination signal consisting of five consecutive adenine-thymidine pairs. Brummelkamp et al., supra. The transcription product is cleaved at a site following the second uridine (of the five encoded by the termination sequence), yielding a transcript which resembles the ends of synthetic siRNAs, which also contain nucleotide overhangs. Sequences to be transcribed into shRNA are cloned into such a vector such that they will generate a transcript comprising a first sequence complementary to a portion of a mRNA target (e.g., a mRNA encoding a lysyl oxidase-type enzyme), separated by a short spacer from a second sequence comprising the reverse complement of the first sequence. The resulting transcript folds back on itself to form a stem-loop structure, which mediates RNA interference (RNAi).

Another suitable siRNA expression vector encodes sense and antisense siRNA under the regulation of separate pol III promoters. Miyagishi et al. (2002) Nature Biotech. 20:497-500. The siRNA generated by this vector also includes a five thymidine (T5) termination signal.

siRNAs, shRNAs and/or vectors encoding them can be introduced into cells by a variety of methods, e.g., lipofection. Vector-mediated methods have also been developed. For example, siRNA molecules can be delivered into cells using retroviruses. Delivery of siRNA using retroviruses can provide advantages in certain situations, since retroviral delivery can be efficient, uniform and immediately selects for stable “knock-down” cells. Devroe et al. (2002) BMC Biotechnol. 2:15.

Recent scientific publications have validated the efficacy of such short double stranded RNA molecules in inhibiting target mRNA expression and thus have clearly demonstrated the therapeutic potential of such molecules. For example, RNAi has been utilized for inhibition in cells infected with hepatitis C virus (McCaffrey et al. (2002) Nature 418:38-39), HIV-1 infected cells (Jacque et al. (2002) Nature 418:435-438), cervical cancer cells (Jiang et al. (2002) Oncogene 21:6041-6048) and leukemic cells (Wilda et al. (2002) Oncogene 21:5716-5724).

Nucleic Acid Inhibitors of LOXL2

Construction of siRNA and shRNA molecules, and vectors encoding them, are well within the skill of the art, as outlined above. In addition siRNA and shRNA molecules targeted to any given nucleotide sequence are readily available commercially, for example from Invitrogen Corp. (Carlsbad, Calif.) and Sigma-Aldrich Corp. (St Louis, Mo.).

Exemplary siRNA sequences that can be used for inhibition of LOXL2 are as follows:

| | |
|---|---|
| UGGAGUAAUCGGAUUCUGCAACCUC | (SEQ ID NO: 14) |
| UCAACGAAUUGUCAAAUUUGAACCC | (SEQ ID NO: 15) |

Exemplary shRNA sequences that can be used for inhibition of LOXL2 are as follows:

(SEQ ID NO: 16)
CCGGCGATTACTCCAACAACATCATCTCGAGATGATGTTGTTGGAGTAAT
CGTTTTTG
(SEQ ID NO: 17)
CCGGGAAGGAGACATCCAGAAGAATCTCGAGATTCTTCTGGATGTCTCCT
TCTTTTTG

The targeting sequence (i.e., the sequence complementary to LOXL2 sequences) in both of the shRNA sequences listed above is underlined.

Methods for Modulating Expression of Lysyl Oxidase-Type Enzymes

Another method for modulating the activity of a lysyl oxidase-type enzyme is to modulate the expression of its encoding gene, leading to lower levels of activity if gene expression is repressed, and higher levels if gene expression is activated. Modulation of gene expression in a cell can be achieved by a number of methods.

For example, oligonucleotides that bind genomic DNA (e.g., regulatory regions of a lysyl oxidase-type gene) by strand displacement or by triple-helix formation can block transcription, thereby preventing expression of a lysyl oxidase-type enzyme. In this regard, the use of so-called “switch back” chemical linking, in which an oligonucleotide recognizes a polypurine stretch on one strand on one strand of its target and a homopurine sequence on the other strand, has been described. Triple-helix formation can also be obtained using oligonucleotides containing artificial bases, thereby extending binding conditions with regard to ionic strength and pH.

Modulation of transcription of a gene encoding a lysyl oxidase-type enzyme can also be achieved, for example, by introducing into cell a fusion protein comprising a functional domain and a DNA-binding domain, or a nucleic acid encoding such a fusion protein. A functional domain can be, for example, a transcriptional activation domain or a transcriptional repression domain. Exemplary transcriptional activation domains include VP16, VP64 and the p65 subunit of NF-κB; exemplary transcriptional repression domains include KRAB, KOX and v-erbA.

In certain embodiments, the DNA-binding domain portion of such a fusion protein is a sequence-specific DNA-binding domain that binds in or near a gene encoding a lysyl oxidase-type enzyme, or in a regulatory region of such a gene. The DNA-binding domain can either naturally bind to a sequence at or near the gene or regulatory region, or can be engineered to so bind. For example, the DNA-binding domain can be obtained from a naturally-occurring protein that regulates expression of a gene encoding a lysyl oxidase-type enzyme. Alternatively, the DNA-binding domain can be engineered to bind to a sequence of choice in or near a gene encoding a lysyl oxidase-type enzyme or in a regulatory region of such a gene.

In this regard, the zinc finger DNA-binding domain is useful, inasmuch as it is possible to engineer zinc finger proteins to bind to any DNA sequence of choice. A zinc finger binding domain comprises one or more zinc finger structures. Miller et al. (1985) EMBO J. 4:1609-1614; Rhodes (1993) Scientific American, February: 56-65; U.S. Pat. No. 6,453,242. Typically, a single zinc finger is about 30 amino acids in length and contains four zinc-coordinating amino acid residues. Structural studies have demonstrated that the canonical (C₂H₂) zinc finger motif contains two beta sheets (held in a beta turn which generally contains two zinc-coordinating cysteine residues) packed against an alpha helix (generally containing two zinc coordinating histidine residues).

Zinc fingers include both canonical C₂H₂ zinc fingers (i.e., those in which the zinc ion is coordinated by two cysteine and two histidine residues) and non-canonical zinc fingers such as, for example, C₃H zinc fingers (those in which the zinc ion is coordinated by three cysteine residues and one histidine residue) and C₄ zinc fingers (those in which the zinc ion is coordinated by four cysteine residues). Non-canonical zinc fingers can also include those in which an amino acid other than cysteine or histidine is substituted for one of these zinc-coordinating residues. See e.g., WO 02/057293 (Jul. 25, 2002) and US 2003/0108880 (Jun. 12, 2003).

Zinc finger binding domains can be engineered to have a novel binding specificity, compared to a naturally-occurring zinc finger protein; thereby allowing the construction of zinc finger binding domains engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. Engineering methods include, but are not limited to, rational design and various types of empirical selection methods.

Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,030,215; 7,067,617; U.S. Patent Application Publication Nos. 2002/0165356; 2004/0197892; 2007/0154989; 2007/0213269; and International Patent Application Publication Nos. WO 98/53059 and WO 2003/016496.

Exemplary selection methods, including phage display, interaction trap, hybrid selection and two-hybrid systems, are disclosed in U.S. Pat. Nos. 5,789,538; 5,925,523; 6,007,988; 6,013,453; 6,140,466; 6,200,759; 6,242,568; 6,410,248; 6,733,970; 6,790,941; 7,029,847 and 7,297,491; as well as U.S. Patent Application Publication Nos. 2007/0009948 and 2007/0009962; WO 98/37186; WO 01/60970 and GB 2,338,237.

Enhancement of binding specificity for zinc finger binding domains has been described, for example, in U.S. Pat. No. 6,794,136 (Sep. 21, 2004). Additional aspects of zinc finger engineering, with respect to inter-finger linker sequences, are disclosed in U.S. Pat. No. 6,479,626 and U.S. Patent Application Publication No. 2003/0119023. See also Moore et al. (2001a) Proc. Natl. Acad. Sci. USA 98:1432-1436; Moore et al. (2001b) Proc. Natl. Acad. Sci. USA 98:1437-1441 and WO 01/53480.

Further details on the use of fusion proteins comprising engineered zinc finger DNA-binding domains are found, for example, in U.S. Pat. Nos. 6,534,261; 6,607,882; 6,824,978; 6,933,113; 6,979,539; 7,013,219; 7,070,934; 7,163,824 and 7,220,719.

Additional methods for modulating the expression of a lysyl oxidase-type enzyme include targeted mutagenesis, either of the gene or of a regulatory region that controls expression of the gene. Exemplary methods for targeted mutagenesis using fusion proteins comprising a nuclease domain and an engineered DNA-binding domain are provided, for example, in U.S. patent application publications 2005/0064474; 2007/0134796; and 2007/0218528.

Formulations, Kits and Routes of Administration

Therapeutic compositions comprising compounds identified as modulators of the activity of a lysyl oxidase-type enzyme (e.g., inhibitors or activators of a lysyl oxidase-type enzyme such as LOXL2) are also provided. Such compositions typically comprise the modulator and a pharmaceutically acceptable carrier. Supplementary active compounds can also be incorporated into the compositions. Modulators, particularly inhibitors, of the activity of LOXL2 are useful, for example, in combination with an anti-neoplastic agent because inhibition of LOXL2 normalizes tumor vasculature and increases perfusion of a tumor, thereby allowing better penetration of the anti-neoplastic agent into the tumor. Accordingly, therapeutic compositions as disclosed herein can contain both a modulator of the activity of a lysyl oxidase-type enzyme and an anti-neoplastic agent, such as a chemotherapeutic drug or a therapeutic biologic. In additional embodiments, therapeutic compositions comprise a therapeutically effective amount of a modulator of the activity of a lysyl oxidase-type enzyme, but do not contain an anti-neoplastic agent, and the compositions are administered separately from the anti-neoplastic agent.

As used herein, the term “therapeutically effective amount” or “effective amount” refers to an amount of a therapeutic agent that when administered alone or in combination with another therapeutic agent to a cell, tissue, or subject (e.g., a mammal such as a human or a non-human animal such as a primate, rodent, cow, horse, pig, sheep, etc.) is effective to prevent or ameliorate the disease condition or the progression of the disease. A therapeutically effective dose further refers to that amount of the compound sufficient to result in full or partial amelioration of symptoms, e.g., treatment, healing, prevention or amelioration of the relevant medical condition, or an increase in rate of treatment, healing, prevention or amelioration of such conditions. A therapeutically effective amount of, for example, an inhibitor of the activity of LOXL2 varies with the type of disease or disorder, extensiveness of the disease or disorder, and size of the organism suffering from the disease or disorder.

The therapeutic compositions disclosed herein are useful for, inter alia, reducing tumor growth. Accordingly, a “therapeutically effective amount” of a modulator (e.g., inhibitor) of the activity of a lysyl oxidase-type enzyme (e.g., LOXL2) ^{_{an amount that results in arrest or reduction of tumor growth. For example, when the inhibitor of LOXL}}2 is an antibody and the antibody is administered in vivo, normal dosage amounts may vary from about 10 ng/kg to up to 100 mg/kg of mammal body weight or more per day, for example, about 1 μg/kg/day to 50 mg/kg/day, optionally about 100 μg/kg/day to 20 mg/kg/day, 500 μg/kg/day to 10 mg/kg/day, or 1 mg/kg/day to 10 mg/kg/day, depending upon, e.g., body weight, route of administration, severity of disease, etc.

When a modulator of the activity of a lysyl oxidase-type enzyme (e.g., LOXL2) ^{_{used in combination with an anti-neoplastic agent, one can also refer to the therapeutically effective dose of the combination, which is the combined amounts of the modulator and the anti-neoplastic agent that result in arrest or reduction of tumor growth, whether administered in combination, serially or simultaneously. More than one combination of concentrations can be therapeutically effective.}}

Various pharmaceutical compositions and techniques for their preparation and use are known to those of skill in the art in light of the present disclosure. For a detailed listing of suitable pharmacological compositions and techniques for their administration one may refer to the detailed teachings herein, which may be further supplemented by texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The disclosed therapeutic compositions further include pharmaceutically acceptable materials, compositions or vehicle, such as a liquid or solid filler, diluent, excipient, solvent or encapsulating material, i.e., carriers. These carriers are involved in transporting the subject modulator from one organ, or region of the body, to another organ, or region of the body. Each carrier should be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Another aspect of the present disclosure relates to kits for carrying out the administration of a modulator of the activity of a lysyl oxidase-type enzyme (e.g., LOXL2). Another aspect of the present disclosure relates to kits for carrying out the combined administration of a modulator of the activity of a lysyl oxidase-type enzyme and an anti-neoplastic agent. In one embodiment, a kit comprises an inhibitor of the activity of a lysyl oxidase-type enzyme (e.g. an inhibitor of LOX or LOXL2) formulated in a pharmaceutical carrier, optionally containing at least one anti-neoplastic agent, formulated as appropriate, in one or more separate pharmaceutical preparations.

The formulation and delivery methods can be adapted according to the site(s) of a tumor and state of tumor growth. Exemplary formulations include, but are not limited to, those suitable for parenteral administration, e.g., intravenous, intra-arterial, intra-ocular, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compounds of the disclosure will vary according to the extent and severity of the need for treatment, the activity of the administered composition, the general health of the subject, and other considerations well known to the skilled artisan.

In additional embodiments, the compositions described herein are delivered locally. Localized delivery allows for the delivery of the composition non-systemically, for example, directly to a tumor, reducing the body burden of the composition as compared to systemic delivery. Such local delivery can be achieved, for example, through the use of various medically implanted devices including, but not limited to, stents and catheters, or can be achieved by injection or surgery. Methods for coating, implanting, embedding, and otherwise attaching desired agents to medical devices such as stents and catheters are established in the art and contemplated herein.

Local delivery to the eye can be achieved, for example, by intra-ocular injection or by application of eye drops.

Combination Therapies

Inhibition of LOXL2 results in the formation of fewer vessels, but increased perfusion, in a tumor. Because of this increased perfusion resulting from LOXL2 inhibition, inhibitors of LOXL2, as disclosed herein, will find use in combination with existing anti-neoplastic agents (e.g., chemotherapeutic drugs) to improve access of the agent to the tumor.

Anti-Neoplastic Agents

For the purposes of the present disclosure, an “anti-neoplastic agent” is any substance used to treat cancer. This includes treatments to a primary tumor (e.g., to inhibit tumor growth), treatments to reduce invasiveness of a primary tumor, and treatments to inhibit metastasis. Anti-neoplastic agents include chemotherapeutic agents (e.g., small organic molecules, generally with a molecular weight less than 1 kDa), therapeutic proteins (e.g., antibodies) and therapeutic nucleic acids. Anti-neoplastic agents often, though not exclusively, block one or more aspects of cell growth and/or proliferation (e.g., they can be cytostatic and/or cytotoxic).

The term neoplastic is understood to mean of, relating to, or having the characteristics of a neoplasm. A neoplasm (literally “new growth”) is a cell or group of cells that is not subject to normal cellular growth controls. Neoplasms can include benign and malignant tumors, as well as myeloproliferative disorders, in which the neoplastic cells can be dispersed, rather than clustered in a benign mass or tumor. Similarly, neoplasia is the abnormal state characterized by the initiation, growth, development and spread (e.g., metastasis) of a tumor.

As used herein the term “chemotherapeutic agent” or “chemotherapeutic” (or “chemotherapy”, in the case of treatment with a chemotherapeutic agent) is meant to encompass any non-proteinaceous (i.e., non-peptidic), non-nucleic acid chemical compound useful in the treatment of cancer.

Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (e.g., bullatacin and bullatacinone); camptothecin (including synthetic analogues topotecan and irinotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g. calicheamicin, especially calicheamicin gamma1I and calicheamicin phiI1, see, e.g., Angew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubincin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-trichlorotriethylamine; trichothecenes (e.g., T-2 toxin, verracurin A, roridin A and anguidine); urethane; vindesine; dacarbazine; mannomustine; mitobronitol; mitolactol; pipobroman; gacytosine; cytosine arabinoside (“Ara-C”); cyclophosphamide; thiopeta; taxoids, e.g. paclitaxel (TAXOL™, Bristol Meyers Squibb Oncology, Princeton, N.J.) and docetaxel (TAXOTERE™., Rhone-Poulenc Rorer, Antony, France); chlorambucil; gemcitabine (Gemzar™); 6-thioguanine; mercaptopurine; methotrexate; platinum analogs such as cisplatin (DDP) and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above.

In some embodiments, an anti-neoplastic agent is a tyrosine kinase inhibitor. For example, ZD1839 (Iressa™ of AstraZeneca K.K.) shows a competitive effect for ATP in ATP binding site of EGFR (epidermal growth factor receptor) tyrosine kinase, and inhibits tyrosine kinase activity by inhibiting autophosphorylation of tyrosine kinase. Another inhibitor of EGFR tyrosine kinase activity is erlotinib (N-(3-ethynylphenyl)-6,7-bis(2-methoxyethoxy)quinazolin-4-amine). Imatinib mesylate (GLEEVEC™, formerly STI-571) can inhibit the tyrosine kinase activity of both BCR-Abl and c-kit. Sorafenib (Nexavar™) is a small molecular inhibitor of Raf kinase, PDGF (platelet-derived growth factor), VEGF receptor 2 & 3 kinases and c-Kit.

Anti-neoplastic agents can also include therapeutic biologics (e.g., therapeutic antibodies), including those raised against tumor antigens or antigens associated with myelodysplasia. Therapeutic antibodies include antibody fragments, as discussed above, monoclonal antibodies, chimeric antibodies and humanized antibodies. Exemplary therapeutic antibodies include the following. IMC-C225 or cetuximab (Erbitux™), is an EGFR-targeted monoclonal antibody that recognizes the receptor portion of EGFR on the cell surface and inhibits the autophosphorylation of EGFR; thereby inhibiting its tyrosine kinase activity. Herceptin (trastuzumab) is a monoclonal antibody directed against the Her2/Neu protein (which is homologous to EGFR and whose overexpression is associated with more aggressive disease and poorer prognosis, particularly in breast cancers). Rituximab (RITUXAN™) is an antibody raised against the CD20 protein on lymphoma cells and which selectively depletes normal and malignant CD20⁺ pre-B and mature B cells. Alemtuzumab (CAMPATHυ) is a monoclonal antibody that specifically targets the CD52 antigen found on B and T lymphocytes; it is used for the treatment of chronic lymphocytic leukemia (CLL) and lymphoma. Gemtuzumab zogamicin (MYLOTARG™) is an antibody conjugate that combines a specific antibody directed against CD33 with a chemotherapeutic drug (zogamicin), and is indicated for the treatment of relapsed adult acute myelocytic leukemia.

### EXAMPLES

### Example 1

### Inhibition of LOXL2 Suppresses bFGF-Induced Cell Migration and Tube Formation in Endothelial Cells

To determine whether LOXL2 activity contributes to bFGF-induced migration of human umbilical vein endothelial cells (HUVEC), experiments were conducted in which expression of LOXL2 was inhibited using a LOXL2-targeted shRNA, and LOXL2 activity was inhibited using an anti-LOXL2 antibody.

HUVEC were isolated from umbilical veins and cultured as described. Gospodarowicz et al. (1983) J. Cell Biol. 97:1677-1685; Kigel et al. (2008) PLoS One 3:e3278. Recombinant human bFGF was produced and purified according to Tessler et al. (1990) J. Cell. Physiol. 145:310-317. For inhibition of LOXL2 expression by shRNA, lentiviruses directing expression of a LOXL2-targeted shRNA and a control shRNA were generated and used to infect HUVEC as described. Brekhman et al. (2009) BMC Cancer 9:415. The anti-LOXL2 monoclonal antibody (AB0023) has been described. See, e.g., Rodriguez et al. (2010) J. Biol. Chem. 285:20964-20974 and United States Patent Application Publication No. 2009/0053224 (Feb. 26, 2009).

For migration assays, HUVEC were seeded in transwell culture dishes (8 μm pores, 3×10⁴ cells/well) coated with fibronectin (10 μg/ml, Biological Industries) in 200 μl of M199 medium containing 20% fetal calf serum. For experiments involving shRNA expression, the HUVEC were infected with the shRNA-encoding lentiviruses described above. For antibody inhibition experiments, 10 μg/ml of either AB0023 or a non-targeting control antibody AC-1 was included in the culture. After 5 hours, cells that had migrated were fixed for 20 minutes with 4% paraformaldehyde, washed three times with PBS, stained with 0.05% crystal violet for 10 minutes, photographed, and counted.

Tube formation on Matrigel was carried out as described. Varshaysky et al. (2008) Cancer Res. 68:6922-2931. For shRNA inhibition HUVEC, infected with either shLOXL2-encoding or control lentivirus, were seeded in Matrigel-coated 48 well tissue culture plates (1.6×10⁴ cells/well) and incubated with growth medium supplemented with bFGF (5 ng/ml) for 24 hours. For antibody inhibition, HUVEC were seeded in Matrigel-coated 48 well tissue culture plates at a density of 1.6×10⁴ cells/well in the presence of either AB0023 or control antibody AC-1 at a concentration of 1 ug/ml, and incubated with growth medium supplemented with bFGF (5 ng/ml) for 24 hours. Phase-contrast images were photographed at 63× magnification and bifurcations-per-field of tube structures in microscopic fields were quantified by manual counting.

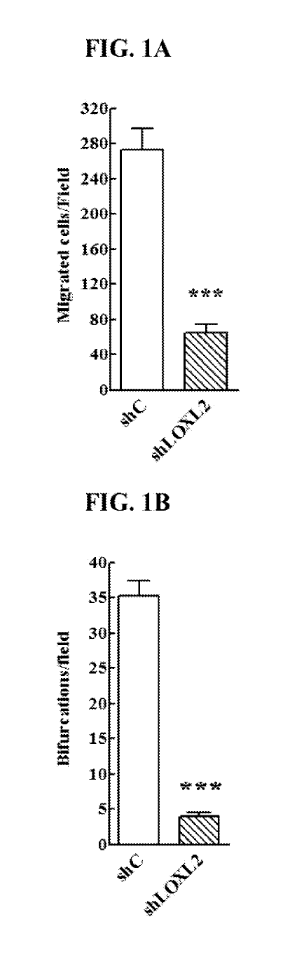

The results of these studies indicated that treatment of HUVEC with a LOXL2-targeted shRNA inhibited bFGF-induced migration (FIG. 1A), and reduced the ability of these cells to form tubular networks on Matrigel (FIG. 1B), when compared to HUVEC infected with a virus expressing a control shRNA. Similar inhibitory effects were observed using a second LOXL2-targeted shRNA and a LOXL2-targeted siRNA, each targeted to a different portion of the LOXL2 mRNA sequence.

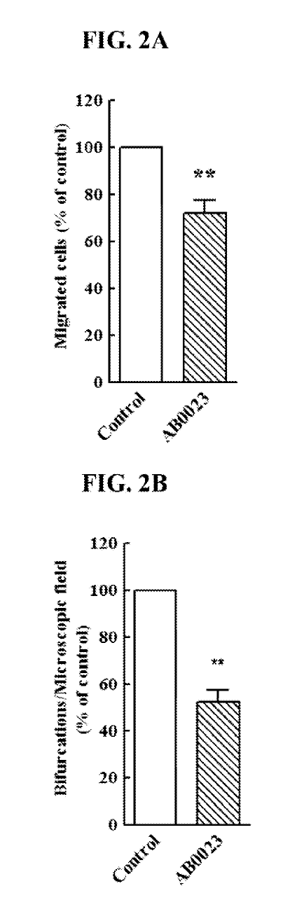

In confirmation of these results, treatment of HUVEC with the anti-LOXL2 antibody AB0023 also inhibited both bFGF-induced cell migration (FIG. 2A) and tube formation (FIG. 2B).

### Example 2

### Inhibition of LOXL2 Suppresses Neovascularization

To determine if inhibition of LOXL2 could modulate angiogenesis in vivo, we examined the effects of the anti-LOXL2 antibody AB0023 on bFGF-induced angiogenesis, using a Matrigel plug assay. Kessler et al. (2004) Cancer Res. 64:1008-1015; Kibbey et al. (1992) J. Natl. Cancer Inst. 84:1633-1638.

For these experiments, either AB0023, at a concentration of 30 mg/kg, or vehicle (PBS+0.01% Tween-20) were injected intraperitoneally (30 mg/kg or equivalent volume) twice weekly into athymic female Ncr:Nu/Nu mice. After one week the mice were injected subcutaneously with 250 or 500 μl Matrigel (BD Biosciences, San Jose, Calif.) supplemented with 100 ng/ml human bFGF (Peprotech, Rocky Hill, N.J.) and 40 or 60 U heparin (Sigma, St. Louis, Mo.). Matrigel plugs were harvested after 10 days, excised together with attached skin, fixed in 10% neutral buffered formalin, and embedded in paraffin. Immunohistochemical analysis was performed on 5 μm serial sections with anti-CD31, anti-CD34, and anti-NG2 antibodies. 2 step sections separated by 800 μM were analyzed per plug. Three representative 10× microscopic fields were selected per section per step, for a total of 6 fields per plug.

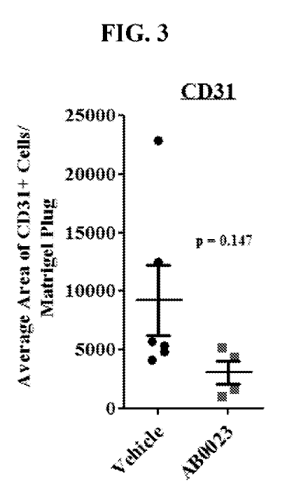

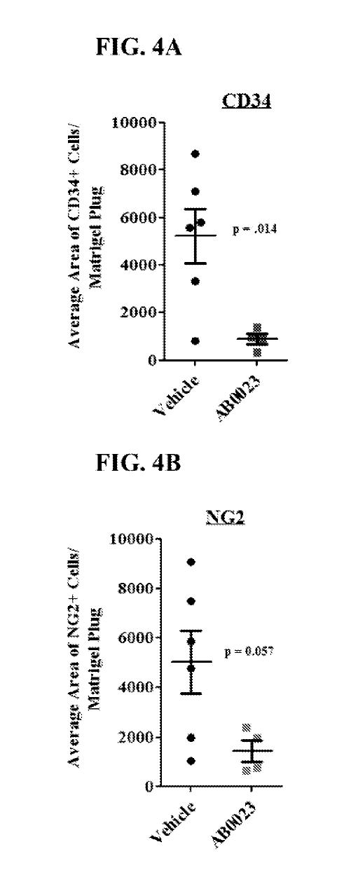

The results of this analysis indicated that systemic application of AB0023 resulted in a significant reduction in the number of blood vessels observed in the Matrigel plugs after 10 days, as assessed by whole plug vessel counting. Quantitation of CD31 in the Matrigel plug cryostat sections was also conducted in this experiment, and showed that CD31 levels were reduced (FIG. 3). In an independent experiment, AB0023 treatment caused a significant reduction in staining for CD34 (FIG. 4A) and for the pericyte marker NG2 (FIG. 4B) in the Matrigel plug system.

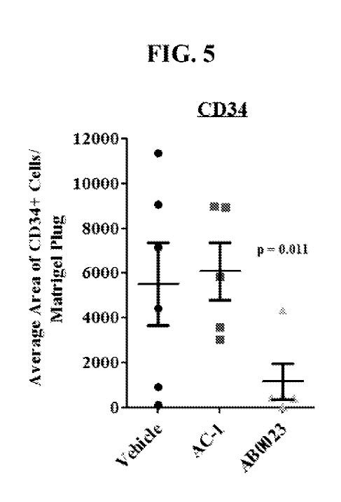

In a further study using the Matrigel plug system, mice were injected with either AB0023, vehicle, or a control antibody (AC-1), that does not bind to LOXL2. Immunohistochemical analysis was performed with anti-CD34 antibody on one representative section from each plug. Three representative 10× fields were selected per section for quantitation. For all IHC analysis of plugs, the mean area of signal was determined for each treatment group using Metamorph software (Molecular Devices). The results, shown in FIG. 5, confirm the loss of CD34 immunoreactivity in implanted plugs after AB0023 treatment, and show that this effect is specific to the anti-LOXL2 antibody, and not simply an artifact of antibody treatment.

### Example 3

### Flow Cytometric Analysis

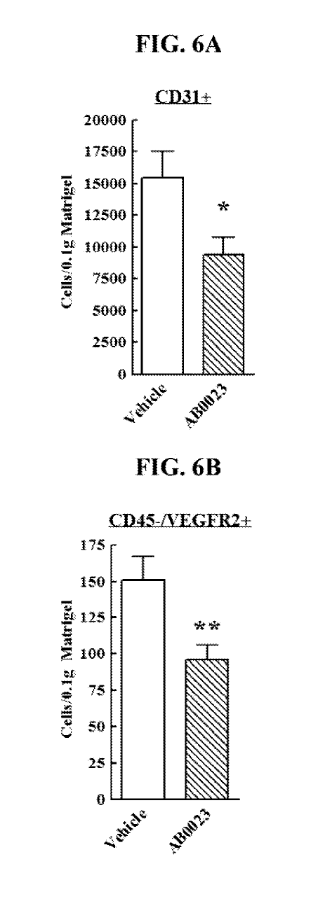

The anti-angiogenic effects of systemic AB0023 administration in the Matrigel plug assay system were also assessed using flow cytometry to assay for cells expressing CD31, and for CD45-negative cells that express the vascular endothelial growth factor receptor VEGFR2 (an angiogenic marker). Analysis was conducted according to the method described by Adini et al. (2009) J. Immunol. Meth. 342:78-81. The results, shown in FIG. 6, indicate that AB0023 treatment significantly reduced the number of both CD31⁺ cells (FIG. 6A) and VEGFR-2⁺/CD45⁻ endothelial cells (FIG. 6B) in plugs harvested 7 days after implantation.

### Example 4

### Tumor Xenograft Model System

SKOV3 ovarian carcinoma cells were obtained from ATCC, Manassas, Va. 5×10⁶ cells, suspended in 50% (v/v) Matrigel, were implanted subcutaneously in female athymic nude mice. After tumors attained a volume of 100 mm³, the mice received twice weekly intra-peritoneal injections of vehicle, anti-LOXL2 antibody AB0023 (15 mg/kg), taxol (5 mg/kg) or a combination of taxol and AB0023. The experiment was terminated when tumors from control (vehicle-treated) animals attained an average volume of 1,000 mm³, at which point the animals were sacrificed, tumors were removed and snap-frozen, and 5 μm thick frozen sections were prepared from the frozen tumors.

Sections were fixed with 4% paraformaldehyde and blocked using Peroxidazed 1 and Background Sniper (Biocare Medical, Concord, Calif.) before incubation with primary antibody. Anti-CA9 antibody (Abcam) was applied and Mach2 HRP-polymer (Biocare Medical) was used as secondary antibody. All sections were dehydrated through a series of ethanol dilutions and xylene, mounted using a resinous medium, and visualized using DAB chromogen.

For assessment of perfusion, the animals were injected intravenously with Hoechst 33342 (40 mg/kg) one minute prior to sacrifice and tumor removal was performed as described by Franco et al. (2006) Cancer Res. 66:3639-3648 and Rijken et al. (2000) Int. J. Radiat. Oncol. Biol. Phys. 48:571-582. 15 μm tumor sections were fixed in acetone for 10 minutes, blocked with PBS containing 10% BSA, and incubated with antibodies directed against CD31 (Pharmingen) or NG2 (Chemicon/Millipore, Temecula, Calif.). CD31 is an endothelial cell marker and NG2 is a marker for pericytes. Bound anti-CD31 was detected with a goat anti-rat CY3-labeled antibody (Jackson Laboratories, Bar Harbor, Me.) and bound NG2 antibodies were detected with a Cy2-labeled anti-rabbit antibody (Jackson Laboratories, Bar Harbor, Me.). The stained sections were visualized using a confocal microscope or a Leica CTR 6000 microscope, and at least five microscopic fields were analyzed in each section. For analysis of perfusion, the coincidence of CD31-staining and Hoechst dye fluorescence was assessed. For determining association of pericytes with blood vessels, the area of NG2-stained cells in association with CD31-stained vessels was determined using Image-Pro® morphometric software (Media Cybernetics, Bethesda, Md.). See, e.g., Weidner (2008) Meth. Enzymology 444:305-323.

Statistical significance was determined using the unpaired Student t-test (normally distributed data) or the Mann-Whitney test (non-normally distributed data), or by ANOVA. Error bars in the figures represent the standard error of the mean. Statistical significance is presented in the figures in the following manner: * indicates p<0.05; ** indicates p<0.01 and *** indicates p<0.001. Experiments were conducted in triplicate.

### Example 5

### Effect of LOXL2 Inhibition on Tumor Growth and Synergism with a Chemotherapeutic

Female athymic nude mice were implanted with SKOV3 ovarian carcinoma cells which, when transplanted, form highly vascularized subcutaneous tumors. Expression of LOXL2 in these tumors was confirmed by immunohistochemistry; it was noted that most of the LOXL2 immunostaining was associated with vascular structures. When the average tumor volume of the population reached 100 mm³, the mice were treated twice weekly with intra-peritoneal injections of the anti-LOXL2 antibody AB0023 (15 mg/kg), taxol (5 mg/kg) or a combination of AB0023 and taxol at the concentrations given above. Control mice received injection of vehicle (PBST). Tumor growth was monitored for 40 days.

The results are shown in FIG. 7. In this model system, treatment of animals with the anti-LOXL2 antibody AB0023 did not inhibit tumor growth; although a change in tumor volume was observed. However, inhibition of LOXL2 activity with the antibody was able to potentiate the growth-inhibitory effect of taxol. Treatment with 5 mg/kg taxol resulted in a small, statistically insignificant inhibition of the increase in tumor volume observed in the control animals. However, the combination of AB0023 and taxol reduced tumor volume by 63% compared to untreated controls (p<0.001) and by 37% compared to animals that had been treated with taxol alone (p<0.05). Thus, inhibition of LOXL2 acted synergistically with a chemotherapeutic to inhibit tumor growth.

### Example 6

### Effect of LOXL2 Inhibition on Vessel Number

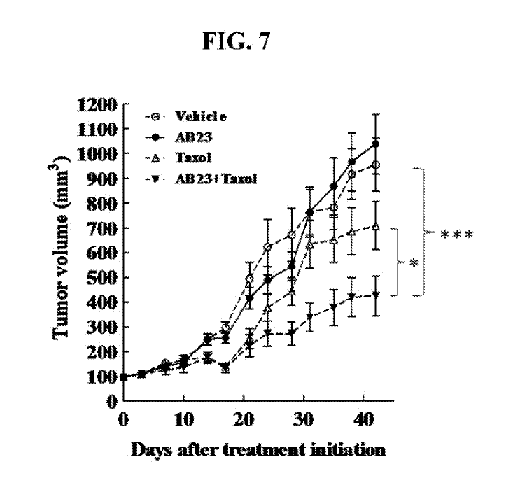

Tumors from animals that had been treated with anti-LOXL2 antibody were compared to tumors from control, vehicle-treated animals with respect to tumor angiogenesis, using CD31 expression as a marker of endothelial cells. FIG. 8 shows that administration of the anti-LOXL2 antibody induced a statistically significant decrease in CD31 immunoreactivity, and consequently, in the concentration of tumor-associated blood vessels.

### Example 7

### Effect of Inhibiting LOXL2 Activity on Tumor Hypoxia

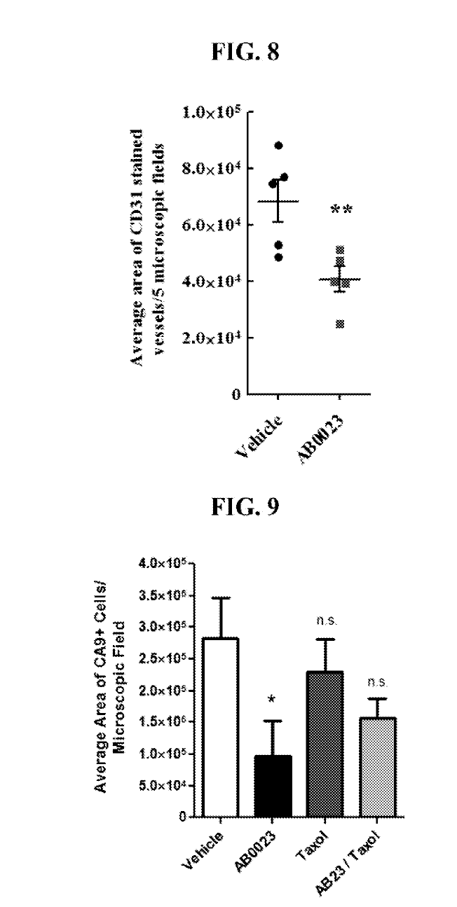

Mice with SKOV3-derived tumors, as described in Example 4, were treated with AB0023 and/or taxol; and the effects on levels of carbonic anhydrase IX (CA9), a marker of hypoxia, were determined. The results are shown in FIG. 9. Although taxol did not have a statistically significant effect on CA9 levels in tumors, inhibition of LOXL2 by treatment with AB0023 resulted in a statistically significant reduction in CA9 levels of 66%. Thus, inhibition of LOXL2 reduces vessel density and reduces hypoxia in tumors.

### Example 8

### Inhibition of LOXL2 Increases Perfusion in Tumor Tissue

Perfusion of the experimental tumors described in Example 4 by tumor-associated vasculature was examined by injecting mice with the dye Hoechst 33342 one minute before excision of tumors, and examining frozen sections of the tumors for Hoechst 33342 fluorescence.

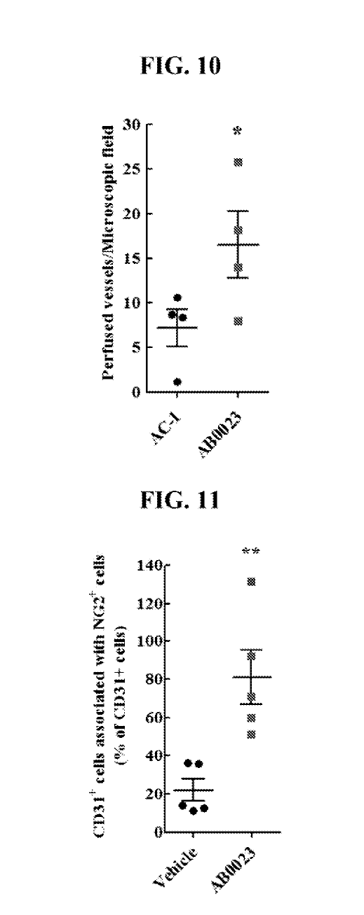

The effect of inhibiting LOXL2 activity on tumor perfusion, using this assay, are shown in FIG. 10, which indicated greater areas of Hoechst staining associated with vasculature (i.e., CD31-positive vessels), and hence better perfusion, in tumors from mice that had been treated with an inhibitor of LOXL2.

### Example 9

### Inhibition of LOXL2 Increases the Number of Blood Vessels Associated with Pericytes in Tumor Tissue

The effects of inhibiting LOXL2 on the number and location of pericytes in tumor tissue were investigated. Immunoreactivity for NG2 was used as a measure of pericyte number. Although the total number of NG2-expressing cells in tumors was not significantly changed by treatment of animals with AB0023; AB0023 treatment did significantly increase the concentration of vessel-associated pericytes in tumors, as measured by correspondence of staining for CD31 and NG2 (FIG. 11). The increased association of pericytes with the tumor microvasculature, in the absence of increases in pericyte number, suggests that inhibition of LOXL2 resulted in recruitment of resident pericytes to the vessels.

Taken together, the increased perfusion, reduced hypoxia, increased pericyte coverage of tumor vessels, and synergism with taxol indicate that inhibition of LOXL2 normalizes tumor vasculature, and promotes enhanced delivery of the chemotherapeutic to the tumor.

1.-6. (canceled)

7. A method for facilitating the delivery of a chemotherapeutic to a tumor, the method comprising:
administering, to a subject with the tumor, an inhibitor of LOXL2.

8. The method of claim 7, wherein the chemotherapeutic is selected from the group consisting of a chemotherapeutic drug and a therapeutic biologic.

9. A method for increasing the efficacy of an anti-neoplastic agent in reducing tumor growth, the method comprising:
administering, to a subject with the tumor, an inhibitor of LOXL2.

10. The method of claim 9, wherein the anti-neoplastic agent is selected from the group consisting of a chemotherapeutic drug, a therapeutic biologic and radiation.

11. A method for increasing the sensitivity of a tumor to a chemotherapeutic, the method comprising:
administering, to a subject with the tumor, an inhibitor of LOXL2.

12. The method of any of claim 7-11 or 20, wherein the inhibitor of LOXL2 is an anti-LOXL2 antibody or antigen-binding fragment thereof.

13. The method of claim 12, wherein the anti-LOXL2 antibody has a heavy-chain amino acid sequence as set forth in SEQ ID NO:3 and a light-chain amino acid sequence as set forth in SEQ ID NO:4.

14.-19. (canceled)

20. The method of claim 11, wherein the chemotherapeutic is selected from the group consisting of a chemotherapeutic drug and a therapeutic biologic.

21. The method of claim 12, wherein the anti-LOXL2 antibody is a humanized antibody.

22. The method of any one of claims 7-11, 20, or 21 wherein the chemotherapeutic is methotrexate, cisplatin, doxorubicin, docetaxel, erlotinib, paclitaxel, paraclitaxel, 5-FU, irinotecan, folinic acid, and gemcitibine.

23. The method of any one of claim 7, 9, or 11, wherein the tumor is selected from the group consisting of: head and neck cancer, bladder cancer, colon cancer, esophageal cancer and breast cancer.
